(19) United States
(12) Patent Application Publication
(10) Pub. No.: US 2008/0039535 A1
(43) Pub. Date: Feb. 14, 2008

(54) METHODS OF IDENTIFYING AGENTS FOR TREATING NEUROLOGICAL DISORDERS

(75) Inventors: Brian Bates, Chelmsford, MA (US); Sheree F. Logue, Yardley, PA (US); Mark Pausch, Rocky Hill, NJ (US)

Correspondence Address: PILLSBURY WINTHROP SHAW PITTMAN, LLP, P.O. BOX 10500, MCLEAN, VA, 22102, US

(73) Assignee: Wyeth, Madison, NJ (US)

(21) Appl. No.: 11/838,843

(22) Filed: Aug. 14, 2007

Related U.S. Application Data
(60) Provisional application No. 60/822,252, filed on Aug. 14, 2006.

(51) Int. Cl. A61K 35/00; A61P 25/18; G01N 33/00

(52) U.S. Cl. 514/789; 800/3

(57) ABSTRACT

Methods of identifying agents for treatment of schizophrenia and related disorders.

### RELATED APPLICATIONS

Priority is claimed to U.S. Provisional Application No. 60/822,252, filed Aug. 14, 2006, which is incorporated by reference herein in its entirety.

### FIELD OF THE INVENTION

The present invention relates to non-human animal models that demonstrate a schizophrenic-like profile responsive to current antipsychotic medications, thereby serving as models for human schizophrenia. The invention also provides methods for identifying therapeutic agents useful for treating schizophrenia and related disorders.

### BACKGROUND OF THE INVENTION

Schizophrenia is a severely debilitating and chronic mental illness found in approximately 1% of the population worldwide. Schizophrenia results from a mix of genetic and environmental factors as indicated by only a 30-40% concordance rate among monozygotic twins (Kringlen, Am. J. Med. Genet. 97:4-11 (2000)). Current antipsychotic medications, typical and atypical, can reduce the severity of schizophrenia symptoms and prevent episodes of relapse, but they suffer from severe limitations, including limited efficacy and serious side effects. In a search for improved therapies, orphan G-protein-coupled receptors (GPCR), having high expression in the mesolimbic and/or mesocortical pathways and commercially available knockout mouse lines, were selected as possible targets for the modulation of the dopamine system. An orphan GPCR, GPR88 (also known as Strg), was identified as having striatal specific expression (Mizushima et al., Genomics 69(3):314-321 (2000)). As described herein, GPR88 knockout mice show a schizophrenia-like behavioral phenotype and have neurochemical characteristics that implicate a role in the dopamine system. Thus, the present invention provides methods of identifying modulators of the dopamine system and agents useful for treating schizophrenia and related neurological disorders.

### SUMMARY OF THE INVENTION

The present invention generally relates to the use of a transgenic animal whose genome lacks a functional GPR88 receptor as an animal model for human schizophrenia. The schizophrenia-like phenotype of the transgenic animal of the present invention is reversed with the treatment of antipsychotics.

The present invention also provides for the use of cells and cell lines derived from a transgenic animal lacking a functional GPR88 receptor in the screening of agents useful in the treatment of schizophrenia. Exemplary cell lines of the present invention include, but are not limited to, CHO, VERO, BHK, HeLa, COS, MDCK, 293, 3T3, and WI38 cells.

The present invention provides for a method of identifying agents that may be useful in the treatment of schizophrenia. In one aspect of the invention, the agent is an agonist to the GPR88 receptor wherein symptoms associated with human schizophrenia are ameliorated.

The present invention also provides for a method of identifying agents useful in the treatment of schizophrenia using a transgenic animal having enhanced dopamine D2 receptor sensitivity to dopamine stimulation. Abnormal dopamine D2 receptor sensitivity may be indicative of several neurological disorders, including, but not limited to, schizophrenia, Parkinson disease, Huntington disease, attention deficit hyperactivity disorder (ADHD), hallucination induced by drug abuse or epilepsy, Tourette's syndrome, restless leg syndrome, obsessive-compulsive disorder (OCD), mania, depression, addiction, and hyperphagia.

The present invention also provides methods for identifying agents useful in the treatment of schizophrenia. In a particular aspect of the invention, the method uses a transgenic animal whose genome lacks a functional GPR88 receptor wherein such a mouse displays a deficit in prepulse inhibition (PPI), an increased sensitivity to apomorphine-induced behaviors, and a decreased level of extracellular dopamine in the striatum. In addition, such an animal displays an increased level of locomotor hyperactivity when stimulated with amphetamines. A deficit in PPI is a hallmark of patients suffering from schizophrenia. In the present invention, apomorphine-induced behaviors include, but are not limited to, locomotor activity, climbing and stereotypy behaviors such as sniffing, licking, biting, and gnawing in mice and rats. In the present invention, extracellular dopamine concentration levels in the striatum can be measured in vivo by, but not limited to, microdialysis, single unit recording of neurons, and positron emission tomography, or ex vivo by, but not limited to, high pressure liquid chromatography and differential pulse voltammetry.

The present invention also provides for methods of identifying agents capable of affecting a phenotype of a transgenic animal. For example, a putative agent is administered to the transgenic animal and a response of the transgenic animal to the putative pharmaceutical composition is measured and compared to the response of a “normal” or wild type mouse, or alternatively compared to a transgenic animal control (without pharmaceutical composition administration). The invention further provides the pharmaceutical compositions identified according to such methods. The present invention also provides for methods of identifying pharmaceutical compositions for treating conditions associated with a disruption of the GPR88 receptor gene, such as amelioration of symptoms associated with human schizophrenia.

The present invention further provides a method of identifying agents having an effect on GPR88 receptor expression or function. The method includes administering an effective amount of the agent to the transgenic mouse. The method includes measuring a response of the transgenic mouse, for example, to the pharmaceutical composition, and comparing the response of the transgenic mouse to a control mouse, which may be, for example, a wild type animal or alternatively, a transgenic mouse control. Compounds that may have an effect on GPR88 receptor expression or function may also be screened against cells in cell-based assays, for example, to identify such compounds.

The invention further provides methods of treating diseases or conditions associated with a disruption in a GPR88 receptor gene, and more particularly, to a disruption in the expression or function of the GPR88 receptor gene. In one aspect of the invention, the methods involve treating diseases or conditions associated with a disruption in the GPR88 receptor gene's expression or function, including administering to a subject in need, a therapeutic pharmaceutical composition that effects GPR88 receptor expression or function. For example the method may comprise administration of a therapeutically effective amount of a natural, synthetic, semi-synthetic, or recombinant GPR88 receptor gene, GPR88 receptor gene products or fragments thereof as well as natural, synthetic, semi-synthetic or recombinant analogs.

### BRIEF DESCRIPTION OF THE FIGURES

FIG. 1: Preparation of GPR88 Knockout Mice. GPR88 KO mice were prepared as described in Example 1. FIG. 1 is a Northern blot showing absence of gpr88 mRNA in cerebral cortex (ctx), striatum (str), and cerebellum (cb) of GPR88 knockout (KO) mice.

FIGS. 2A and 2B: Effect of GPR88 on Startle Habituation Comparing WT and KO mice (n=10). FIGS. 2A and 2B are line graphs showing the mean +/−sem average startle response of GPR88 knockout and WT male (FIG. 2A) and female mice (FIG. 2B). The y-axes depicts the average startle movement of the mice after 10 consecutive 110 db acoustic bursts. Each set of 10 consecutive 110 db acoustic bursts is shown on the x-axes. The startle response was measured both for male GPR88 knockout (closed circles) and wild type (closed squares) mice, and female GPR88 knockout (open circles) and wild type (open squares) mice.

FIGS. 3A and 3B: Effect of GPR88 on Startle Sensitivity Comparing WT and KO mice (n=10). FIGS. 3A and 23B are line graphs showing the mean +/−sem average startle sensitivity of GPR88 knockout and WT male (FIG. 3A) and female (FIG. 3B) mice with one of eight different startle intensities (ranging from 90 to 120 dB in 5 dB increments). The y-axes depict the average startle movement of the mice at 1 of 8 startle intensities presented 5 times each over the course of the session. The eight different startle intensities are presented on the x-axes. The startle sensitivity was measured in both male GPR88 knockout (closed circles) and wild type (closed squares) mice, and female GPR88 knockout (open circles) and wild type (open squares) mice.

FIGS. 4A and 4B: Effect of GPR88 on Prepulse Inhibition of Startle Response Comparing WT (male=10; female=8) and KO mice (male=11; female=11). FIGS. 4A and 4B are bar graphs showing the percentage prepulse inhibition of GPR88 knockout and WT male (FIG. 4A) and female mice (FIG. 4B). The y-axes depict the percentage prepulse inhibition, which is calculated as 100 minus the ratio of the average startle response observed at each prepulse+pulse trial relative to the pulse alone stimulus, times 100. The x-axes indicate the three different prepulse intensity (dB) levels relative to the background. The background noise level was measured at 64 dB. Three prepulses at either 69, 76, or 84 dB were used in conjunction with a 118 dB pulse that followed the initial prepulse after 100 milliseconds. The percentage prepulse inhibition was measured both for male GPR88 knockout (solid bars) and wild type (open bars), and female GPR88 knockout (striped bars) and WT (solid bars) mice.

FIGS. 5A and 5B: Effect of Haloperidol on Prepulse Inhibition on Startle Response in WT (n=18) and GPR88 KO mice (n=18). FIGS. 5A and 5B are bar graphs showing the effect of haloperidol on prepulse inhibition. The percentage prepulse inhibition is calculated as 100 minus the ratio of the average startle response observed at each prepulse+pulse trial relative to the pulse alone with stimulus, times 100. The y-axes indicate the percentage prepulse inhibition across the three prepulse levels of either 69, 76, or 84 dB as used in conjunction with a 118 dB pulse that followed the initial prepulse after 100 milliseconds. The background noise level was measured at 64 dB. FIG. 5A indicates PPI levels of male WT and GPR88 KO mice treated with either 0.54 mg/kg of haloperidol or vehicle. FIG. 5B indicates female WT and GPR88 KO mice treated with either 0.54 mg/kg of haloperidol or vehicle. The percentage prepulse inhibition was measured for both haloperidol treated male (solid bars) and female (striped bars) GPR88 knockout mice, and untreated (open bars) male and (solid bars) female GPR88 knockout mice.

FIG. 6: Effect of Risperidone on Prepulse Inhibition on Startle Response in male GPR88 KO (n=10) and WT mice (n=10). FIG. 6 is a bar graph showing the percentage prepulse inhibition of risperidone treated vs. untreated WT and GPR88 knockout male mice. The y-axis measures the percentage prepulse inhibition. The x-axis indicates the average prepulse inhibition across the three prepulse levels of either 69, 76, or 84 dB as used in conjunction with a 118 dB pulse that followed the initial prepulse after 100 milliseconds. The background noise level was measured at 64 dB. The percentage prepulse inhibition was measured both for risperidone treated male GPR88 knockout and WT mice (solid bars), and untreated GPR88 knockout and WT mice (open bars).

FIGS. 7A-7D: Effect of GPR88 on Apomorphine-Induced Climbing and Stereotypy Comparing WT and KO mice (n=10). FIGS. 7A-7D depict four bar graphs showing the average total score of each WT and GPR88 KO mouse. The y-axes depict the WT and GPR88 KO mice average scores for their ability to demonstrate climbing behavior (0=no climbing, all four feet on the bottom of cage, 1=two feet up on wire cage, 2=all four feet up on wire cage) or stereotypy behavior like sniffing, licking and gnawing (1 for each characteristic expressed by mouse, 0 for each characteristic absent) as measured every 5 minutes over a 30-minute period. The x-axes indicate the dosage amount of apomorphine given to the mice. The behavioral assessment was conducted over three conditions in succession. First a subcutaneous vehicle (0.25% TWEEN® 80) was used to establish a baseline. Second, a dose of 0.3 mg/kg of apomorphine followed. Third, an evaluation of the response to 1 mg/kg of apomorphine was given. FIG. 7A shows the climbing scores at each prescribed dose of apomorphine of WT (open bars) and GPR88 knockout (black bars) male mice. FIG. 7B shows the stereotypy scores at each prescribed dose of apomorphine of male WT (dotted bars) and GPR88 KO (cross-hatched bars) mice. FIG. 7C shows the climbing scores at each prescribed dose of apomorphine of WT (solid bars) and GPR88 knockout (dotted bars) female mice. FIG. 7D shows the stereotypy scores at each prescribed dose of apomorphine of WT (vertical striped bars) and GPR88 KO female (horizontal striped bars) mice.

FIGS. 8A-8D: Effect of Haloperidol on Apomorphine-Induced Climbing and Stereotypy in WT (n=10) and GPR88 KO Mice (n=10). FIGS. 8A-8D are line graphs showing the average percentage of climbing behavior and stereotypy behavior, e.g., sniffing, licking, and gnawing, exhibited by WT and GPR88 KO male and female mice that are either dosed with vehicle or haloperidol. Separate groups of the WT and GPR88 KO mice were either given 0.3, 0.54, or 1.0 mg/kg of haloperidol prior to apomorphine induction as indicated on the x-axes. The y-axes provide the percentage of climbing or stereotypy behavior as scored in FIGS. 8A-8D. Data are mean +/−sem of the percentage of vehicle control group. FIG. 8A shows male WT (closed squares) and GPR88 knockout mice (closed circles) treated with either 0.3, 0.54, or 1 mg/kg of haloperidol prior to apomorphine induction of climbing behavior relative to male mice (WT and GPR88 knockout) treated with only vehicle prior to apomorphine induction. FIG. 8B shows male WT (closed squares) and GPR88 knockout mice (closed circles) treated with either 0.3, 0.54, or 1 mg/kg of haloperidol prior to apomorphine induction of stereotypy behavior relative to the male mice (WT and GPR88 knockout) treated only with vehicle prior to apomorphine induction. FIG. 8C shows female WT (open squares) and GPR88 knockout mice (closed squares) treated with either 0.3, 0.54, or 1 mg/kg of haloperidol prior to apomorphine induction of climbing behavior relative to the female mice (WT and GPR88 knockout) treated only with vehicle prior to apomorphine induction. FIG. 8D shows female WT (open squares) and GPR88 knockout mice (open circles) treated with either 0.3, 0.54, or 1 mg/kg of haloperidol prior to apomorphine induction of stereotypy behavior relative to the female mice (WT and GPR88 knockout) treated only with vehicle prior to apomorphine induction.

FIGS. 9A and 9B: Haloperidol-induced Catalepsy in WT (n=6) and GPR88 KO (n=6) mice. FIGS. 9A and 9B are bar graphs demonstrating the percentage maximum catalepsy as a function of time for male WT and GPR88 KO mice, (FIG. 9A) and female (FIG. 9B). For induction of catalepsy, haloperidol (1 mg/kg) was administered 30 minutes prior to catalepsy assessment. Thirty, 60, 90, and 120 minutes following drug administration, the mice's forelimbs were draped over a thin rod 1¾″ high. The amount of time in seconds the mice maintained this awkward position was recorded (60 seconds maximum). Maintaining this position was considered catalepsy. Mean time spent in the catalepsy position for the dose at each time was expressed as a percentage of maximum possible catalepsy. Data was subjected to repeated-measures ANOVA followed by post-hoc analysis of least squared means.

FIGS. 10A and 10B: Effect of GPR88 on Spontaneous Activity and Amphetamine-Induced Locomotor Hyperactivity in male and female WT (n=10) and GPR88 KO (n=10) mice. FIGS. 10A and 10B show the average activity of male (FIG. 10A) and female (FIG. 10B) WT and GPR88 KO mice over a 90-minute test session. Mice were injected with amphetamine after being allowed to habituate for 30 minutes. Activity was recorded in 10-minute bins during the 30-minute habituation (spontaneous activity) and during the 60 minutes following amphetamine stimulation (hyperactivity stimulation). Activity counts in 10-minute bins was analyzed by repeated-measures ANOVA followed by post-hoc analysis of least squared means.

FIGS. 11A and 11B: Effect of GPR88 on Spontaneous Activity and Phencyclidine-Induced Locomotor Hyperactivity in male and female WT (n=10) and GPR88 KO (n=10) mice. FIGS. 11A and 11B show the activity as measured in centimeters traveled over a 90 minute period for male WT and GPR88 KO mice (FIG. 11A) and female WT and GPR88 KO mice (FIG. 11B) treated with 1.0 mg/kg, 1.7 mg/kg or 3.0 mg/kg of phencyclidine (PCP). WT (open symbols) and GPR88 KO mice (closed symbols) were injected with three different doses phencyclidine (PCP) after being allowed to habituate for 30 minutes. Activity was recorded in 10-minute bins during the 30-minute habituation (spontaneous activity) and during the 60 minutes following PCP stimulation (hyperactivity stimulation). Activity counts in 10-minute bins was analyzed by repeated-measures ANOVA followed by post-hoc analysis of least squared means. See Example 10. Open and closed squares=1.0 mg/kg PCP; open and closed circles=1.7 mg/kg PCP; open and closed triangles=3.0 mg/kg PCP.

FIGS. 12A-12C: Number, Affinity and Sensitivity of Dopamine D₂ Receptors in GPR88 Mice. FIGS. 12A and 12B show plots of total, non-specific and specific [³H]spiperone binding to striatal membrane preparations from GPR88 KO mice (FIG. 12A) and WT mice (FIG. 12B). Striatal membrane preparations were obtained from a pool of 10 GPR88 KO or WT mice. The estimates of Bₘₐₓ (the number of available binding receptors) and K_{d} (the dissociation constant) were calculated as described in Example 12. No significant differences in K_{d} or Bₘₐₓ values between the genotypes was observed. FIG. 12C, shows a bar graph depicting the percent stimulation of [³⁵S]GTPγS striatal binding due to dopamine agonism for GPR88 KO and WT mice. Striatal membranes were prepared as described in Example 14. [³⁵S]GTPγS striatal binding was determined for each sample in triplicate in the absence and presence of 1 mM dopamine, also as described in Example 14. The difference in the mean of the triplicates for each individual sample (9 GPR88 KO or WT mice) in the absence and presence of dopamine was used to determine the percent stimulation of [³⁵S]GTPγS binding. The percent stimulation for each genotype was plotted as the mean of the individual samples, +/− the standard deviation. [³⁵S]GTPγS binding was significantly greater in the GPR88 KO mice than in the WT mice (p=0.0362).

FIGS. 13A and 13B: Effect of Amphetamine on Striatal Dopamine in male and female GPR88 KO and WT Mice. FIGS. 13A and 13B show the effect of amphetamine on striatal dopamine concentrations in male (FIG. 13A) and female (FIG. 13B) GPR88 KO and WT mice via striatal microdialysis in awake freely moving mice. Mice were dosed with either vehicle (saline) or amphetamine at T=0 minutes. Dialysates collected every 30 minutes for 3 hours.

### DETAILED DESCRIPTION OF THE INVENTION

The present invention provides methods of identifying agents useful in the treatment of schizophrenia and other neurological disorders. Schizophrenia is a severely debilitating and chronic mental illness found in approximately 1% of the population worldwide. Schizophrenia results from a mix of genetic and environmental factors as indicated by only a 30-40% concordance rate among monozygotic twins (Kringlen, Am. J. Med. Genet. 97:4-11 (2000)). There is a developmental aspect to the illness in that the symptoms of schizophrenia typically emerge during adolescence or early adulthood.

Symptoms of schizophrenia are categorized into three category clusters. Positive symptoms include hallucination, delusions, catatonic behaviors and severe thought disorganization. Negative symptoms include flattened affect, apathy, poverty of speech, anhedonia and social withdrawal. Cognitive symptoms include deficits in attention and memory that are apparent as severe deficits in a variety of measures of attention, executive function and memory (Nuechterlein et al., Schizophr. Res. 72:29-39 (2004)).

Although the etiology and pathology of schizophrenia are not fully understood, there is much evidence that disruptions in several neurotransmitter systems are involved in the pathophysiological process leading to the formation of schizophrenic symptoms. The dopamine system was the first system implicated in the disease and has been the most extensively studied. A long history of research has demonstrated the efficacy of D2 receptor antagonism in the alleviation of only the positive symptoms of schizophrenia (Gray, Schizophr. Bull. 24:249-66 (1998)). Thus, while the dopamine D2 receptor is the primary target of all current antipsychotic medications, these medications primarily treat only the positive symptoms of the disease, i.e., hallucination, delusions, and severe thought disorganization.

Dopamine and Dopamine Receptors

In one aspect of the invention, methods of identifying antipsychotic agents rely on enhanced dopamine D2 receptor sensitivity upon dopamine stimulation, as observed in animals lacking a functional GPR88 receptor. The dopamine D2 receptor is one of five subtypes of dopamine receptors. D1 and D5 receptors form the D1-like family of dopamine receptors, while D2, D3 and D4 form the D2-like family of receptors. Activation of the excitatory D1-like family receptors is coupled to increases in cAMP, while the D2-like receptor family activation reduces cAMP and is typically inhibitory (Nicola et al., Annu. Rev. Neurosci. 23:185-215 (2000)). All dopamine receptors are G-protein coupled metabotropic receptors.

The endogenous ligand for dopamine receptors is dopamine. Dopamine, a chemical precursor to epinephrine and norepinephrine, is both a neurotransmitter in the central nervous system (CNS) and a neurohormone affecting the release of prolactin from the pituitary. Upon dopamine binding, dopamine receptors initiate downstream signaling events, which, in turn, initiate gene transcription.

Disruptions to the dopamine system have been strongly linked to schizophrenia and psychosis. One of the strongest pieces of evidence for a dopamine disruption in schizophrenia arises from the ability of D2 receptor antagonists to alleviate schizophrenic symptoms. Similarly, drugs such as amphetamines and cocaine, which are known to greatly increase dopamine levels, can induce psychosis. Based on these facts, dopamine and dopamine receptors likely play in integral role in schizophrenia, psychosis, and wherever dopamine receptors are expressed.

Dopaminergic Projections

Projections of the dopaminergic system can be divided into nigrostriatal, mesolimbic and mesocortical systems (Lindvall, in Chemical Neuroanatomy, Raven Press, New York, pp. 229-55 (1983)). The nigrostriatal system projects from the substantia nigra (SN) to the dorsal striatum and has been identified to be involved in initiation of movement, sensorimotor coordination and cognitive integration. The mesolimbic system projects from the ventral tegmental area (VTA) to limbic structures such as ventral striatum, which includes the nucleus accumbens and the ventral parts of the caudate and putamen, the hippocampus and the amygdala. The mesocortical system projects from the VTA to cortical regions, mostly orbitofrontal, medial prefrontal and cingulate cortices but to some degree to the dorsolateral prefrontal cortex, temporal and parietal cortex. Regulation of motivation, attention and reward are the function of the mesolimbic and mesocortical systems (Mogenson et al., Prog. Neurobiol. 14:69-97 (1980); Horvitz, Neuroscience 96:651-56 (2000); O'Donnell, Behav. Brain Sci. 28:360-61 (2005)). In schizophrenia, the dysfunction of the mesolimbic system, thought to be hyperdopaminergia, is hypothesized to underlie the positive symptoms whereas dysfunction of the mesocortical system, thought to be hypodopaminergia, is hypothesized to underlie the cognitive symptoms.

The current antipsychotic medications, typical and atypical, are effective at reducing the severity of schizophrenia symptoms and at preventing episodes of relapse, but they suffer from severe limitations. Blockade of the D2 receptors, in both typical and atypical antipsychotics, is only effective at reducing, but not fully preventing, the positive symptoms, while having nearly no effect on the negative or cognitive symptoms. Non-dopaminergic actions of the atypical antipsychotics improve some aspects of cognition (Lee et al., Schizophr. Res. 37:1-11 (1999); Meltzer and McGurk, Schizophr. Bull. 25:233-55 (1999); Meltzer et al., Proc. Natl. Acad. Sci. USA 96:13591-93 (1999); Meltzer and Sumiyoshi, Biol. Psychiatry 53:265-67; author reply 267-68 (2003); McGurk et al., Med Gen Med 6:27 (2004)). In fact, the hypodopaminergia resulting from the blockade of the D2 receptors might exacerbate the negative and cognitive symptoms (Abi-Dargham and Laruelle, Eur. Psychiatry 20:15-27 (2005). The typical and atypical antipsychotic medications are also saddled with very serious side effects. For typical antipsychotics, these side effects include extrapyramidal symptoms (EPS) driven through the D2 receptor blockade in the nigrostriatal pathway and hyperprolactinemia related to D2 receptor blockade in the tuberoinfundibular pathways (Kaiser et al., Mol. Psychiatry 7:695-705 (2002); Tauscher et al., Psychopharmacology (Berl.) 162:42-49 (2002); Yamada et al., Synapse 46:32-37 (2002); Haddad and Wieck, Drugs 64:2291-2314 (2004); Matsui-Sakata et al., Drug Metabol. Pharmacokinet. 20:187-1999)). With atypical antipsychotics, the D2 antagonism driven side effects are reduced, but weight gain and diabetes are a tremendous liability (Allison et al., Am. J. Psychiatry 156:1686-96 (1999); Masand, Expert Opin. Pharmacother. 1:377-89 (2000)).

GPR88

The invention is based, in part, on the GPR88 receptor gene and protein. This novel orphan G-coupled protein receptor contains seven transmembrane domains and shares significant homology to other GPCRs (Mizushima et al., Genomics 69:314-21 (2000)). An endogenous ligand for GPR88 has yet to be identified.

Striatum

Because GPR88 expression is primarily in the striatum, the present invention seeks to identify agents that affect functions associated with the striatum. The striatum is a subcortical part of the brain consisting of the caudate nucleus and the putamen. It is part of the basal ganglia. The striatum is best known for its role in the planning and modulation of movement pathways but is also involved in a variety of other cognitive processes involving executive function. Also, it is possible that GPR88 modulates dopamine or D2 receptor expression within the striatum and would accordingly be a focal point for understanding the planning and modulation of movement pathways. Defects within the striatum have been associated with a number of diseases and disorders, including, but not limited to, schizophrenia, Parkinson disease, Huntington disease, attention deficit hyperactivity disorder (ADHD), hallucination induced by drug abuse or epilepsy, Tourette's syndrome, restless leg syndrome, obsessive-compulsive disorder (OCD), mania, depression, drug addiction, or hyperphagia.

Animal Models

The present invention describes cell- and animal-based systems that can be used as models for diseases that affect normal neurological function. Animals of any species, including, but not limited to, mice, rats, rabbits, guinea pigs, pigs, micro-pigs, goats, and non-human primates, e.g., baboons, monkeys, and chimpanzees may be used to generate disease animal models. In addition, cells from humans may be used. These systems may be used in a variety of applications. Such assays may be utilized as part of screening strategies designed to identify agents, such as compounds that are capable of ameliorating disease symptoms. Thus, the animal- and cell-based models may be used to identify agents, therapies and interventions that may be effective in treating disease.

Cell-based systems may be used to identify compounds that may act to ameliorate disease symptoms. For example, such cell systems may be exposed to a compound suspected of exhibiting an ability to ameliorate disease symptoms, at a sufficient concentration and for a time sufficient to elicit such an amelioration of disease symptoms in the exposed cells. After exposure, the cells are examined to determine whether one or more of the disease cellular phenotypes has been altered to resemble a more normal or more wild type, non-disease phenotype.

In addition, animal-based disease systems, such as those described herein, may be used to identify compounds capable of ameliorating disease symptoms. Such animal models may be used as test substrates for the identification of drugs, pharmaceuticals, therapies, and interventions that may be effective in treating a disease or other phenotypic characteristic of the animal. For example, animal models may be exposed to a compound or agent suspected of exhibiting an ability to ameliorate disease symptoms, at a sufficient concentration and for a time sufficient to elicit such an amelioration of disease symptoms in the exposed animals. The response of the animals to the exposure may be monitored by assessing the reversal of disorders associated with the disease. Exposure may involve treating mother animals during gestation of the model animals described herein, thereby exposing embryos or fetuses to the compound or agent that may prevent or ameliorate the disease or phenotype. Neonatal, juvenile, and adult animals can also be exposed.

More particularly, using the animal models of the invention, e.g., transgenic mice, methods of identifying agents, including compounds, are provided on the basis of the ability to affect at least one phenotype associated with a disruption in a GPR88 receptor gene. For example, the method may comprise identifying agents having an effect on GPR88 receptor expression or function. The method includes measuring a physiological response of the animal, for example, to the agent, and comparing the physiological response of such animal to a control animal, wherein the physiological response of the animal comprising a disruption in a GPR88 receptor as compared to the control animal indicates the specificity of the agent. A “physiological response” is any biological or physical parameter of an animal that can be measured. Molecular assays (e.g., gene transcription, protein production and degradation rates), physical parameters (e.g., exercise physiology tests, measurement of various parameters of respiration, measurement of heart rate or blood pressure, measurement of bleeding time), and cellular assays (e.g., immunohistochemical assays of cell surface markers, or the ability of cells to aggregate or proliferate) can be used to assess a physiological response. The transgenic animals and cells of the present invention may be utilized as models for diseases, disorders, or conditions associated with phenotypes relating to a disruption in a GPR88 receptor.

The present invention provides a unique animal model for testing and developing new treatments relating to the behavioral phenotypes. Analysis of the behavioral phenotype allows for the development of an animal model useful for testing, for instance, the efficacy of proposed genetic and pharmacological therapies for human genetic diseases, such as neurological, neuropsychological, or psychotic illnesses.

The animal models which may be used for testing and developing new treatments relating to the behavioral phenotypes described herein include transgenic animals comprising a disruption of the GPR88 locus (see e.g., Genbank Accession Nos. AAH36775 and Q9EPB7). Such “knockout” mice may be characterized as null, i.e., lacking all detectable GPR88 function, or as hypomorphic, i.e., lacking some but not all GPR88 function and which show one or more behavioral phenotypes consistent with a neurological disorder, as described in the Examples.

In a representative aspect of the invention, a GPR88 knockout mouse may be obtained by homozygous breeding within knockout lines originating from an F1 cross of 129 and C57BL/6 supplied by Lexicon Genetics Incorporated (The Woodlands, Tex.), as described in Example 1. See also U.S. Pat. Nos. 5,464,764; 5,487,992; 5,627,059; 5,631,153; and 5,789,215. Mice with complete or partial functional inactivation of the GPR88 gene in all somatic cells may also be generated using standard techniques or site-specific recombination in embryonic stem cell. See Capecchi, Science, 1989, 244: 1288-1292; Thomas & Capecchi, Nature, 1990, 346:847-850; Delpire et al., Nat. Genet., 1999, 22:192-195. A disrupted GPR88 locus may result in an altered level of a GPR88 gene or the expression of a mutated variant of a GPR88 gene.

A transgenic animal in accordance with the present invention may also be prepared using an anti-sense or ribozyme GPR88 construct, driven by a universal or tissue-specific promoter, to reduce levels of GPR88 gene expression in somatic cells, thus achieving a “knock-down” phenotype. The present invention also provides the generation of transgenic animals having conditional or inducible inactivation of GPR88.

The present transgenic animals may include specific “knocked-in” modifications in the GPR88 gene, for example to create an overexpression or dominant negative phenotype. Thus, “knocked-in” modifications include the expression of both wild type and mutated forms of a nucleic acid encoding a GPR88 protein. Knock-in transgenic organisms may be made in any relevant species.

Techniques for the preparation of transgenic animals are known in the art. Exemplary techniques are described in U.S. Pat. No. 5,489,749 (transgenic rats); U.S. Pat. Nos. 4,736,866, 5,550,316, 5,614,396, 5,625,125 and 5,648,061 (transgenic mice); U.S. Pat. No. 5,573,933 (transgenic pigs); U.S. Pat. No. 5,162,215 (transgenic avian species) and U.S. Pat. No. 5,741,957 (transgenic bovine species), the entire contents of each of which are herein incorporated by reference.

GPR88 modulators can be identified by assessing, e.g., startle sensitivity or sensitivity to apomorphine-induced behaviors in wild type and transgenic animals following administration of a test agent. These relevant behavioral indices are described in the examples below.

A statistical analysis of the various behaviors measured can be carried out using any conventional statistical program routinely used by those skilled in the art (such as, for example, “Analysis of Variance” or ANOVA). A “p” value of about 0.05 or less is generally considered to be statistically significant, although slightly higher p values may still be indicative of statistically significant differences. To statistically analyze abnormal behavior, a comparison is made between the behavior of a transgenic animal (or a group thereof) to the behavior of a wild type (WT) mouse (or a group thereof), typically under certain prescribed conditions. “Abnormal behavior” as used herein refers to behavior exhibited by an animal having a disruption in the GPR88 receptor gene, e.g. transgenic animal, which differs from an animal without a disruption in the GPR88 receptor gene, e.g., a wild type mouse. Abnormal behavior consists of any number of standard behaviors that can be objectively measured (or observed) and compared. In the case of comparison, the change should be statistically significant to confirm that there is indeed a meaningful behavioral difference between the knockout animal and the wild type control animal. Examples of behaviors that may be measured or observed include, but are not limited to, ataxia, rapid limb movement, eye movement, breathing, motor activity, cognition, emotional behaviors, social behaviors, hyperactivity, hypersensitivity, anxiety, impaired learning, abnormal reward behavior, and abnormal social interaction, such as aggression.

A series of tests may be used to measure the behavioral phenotype of the animal models of the present invention, including neurophysiological, neurological, and neuropsychological tests to identify abnormal behavior. These tests may be used to measure abnormal behavior relating to, for example, learning and memory, eating, pain, aggression, sexual reproduction, anxiety, depression, schizophrenia, and drug abuse. See e.g., Crawley & Paylor, Hormones and Behavior 31:197-211 (1997).

The social interaction test involves exposing a mouse to other animals in a variety of settings. The social behaviors of the animals (e.g., touching, climbing, and sniffing) are subsequently evaluated. Differences in behaviors can then be statistically analyzed and compared (see e.g., File et al., Pharmacol. Bioch. Behav. 22:941-944 (1985); Holson, Phys. Behav. 37:239-247 (1986)). Exemplary behavioral tests include the following.

The mouse startle response test typically involves exposing the animal to a sensory (typically auditory) stimulus and measuring the startle response of the animal (see e.g., Geyer et al., Brain Res. Bull. 25:485-498 (1990); Paylor and Crawley, Psychopharmacology 132:169-180 (1997)). A prepulse inhibition (PPI) test can also be used, in which the percentage inhibition is measured by “cueing” the animal first with a brief low-intensity prepulse prior to the startle pulse.

The electric shock test generally involves exposure to an electrified surface and measurement of subsequent behaviors such as, for example, motor activity, learning, social behaviors. The behaviors are measured and statistically analyzed using standard statistical tests. (see e.g., Kant et al., Pharm. Bioch. Behav. 20:793-797 (1984); Leidenheimer et al., Pharmacol. Bioch. Behav. 30:351-355 (1988)).

The tail-pinch or immobilization test involves applying pressure to the tail of the animal and/or restraining the animal's movements. Motor activity, social behavior, and cognitive behavior are examples of the areas that are measured. (see e.g., Bertolucci-D'Angio et al., J. Neurochem. 55:1208-1214 (1990)).

The novelty test generally comprises exposure to a novel environment and/or novel objects. The animal's motor behavior in the novel environment and/or around the novel object are measured and statistically analyzed. (see e.g., Reinstein et al., Pharm. Bioch. Behav. 17:193-202 (1982); Poucet, Behav. Neurosci. 103:1009-10016 (1989); Holson et al., Phys. Behav. 37:231-238 (1986)). This test may be used to detect visual processing deficiencies or defects.

The learned helplessness test involves exposure to stresses, for example, noxious stimuli, which cannot be affected by the animal's behavior. The animal's behavior can be statistically analyzed using various standard statistical tests. (see e.g., Leshner et al., Behav. Neural Biol. 26:497-501 (1979)).

Alternatively, a tail suspension test may be used, in which the “immobile” time of the mouse is measured when suspended “upside-down” by its tail. This is a measure of whether the animal struggles, an indicator of depression. In humans, depression is believed to result from feelings of a lack of control over one's life or situation. It is believed that a depressive state can be elicited in animals by repeatedly subjecting them to aversive situations over which they have no control. A condition of “learned helplessness” is eventually reached, in which the animal will stop trying to change its circumstances and simply accept its fate. Animals that stop struggling sooner are believed to be more prone to depression. Studies have shown that the administration of certain antidepressant drugs prior to testing increases the amount of time that animals struggle before giving up.

The Morris water-maze test comprises learning spatial orientations in water and subsequently measuring the animal's behaviors, such as, for example, by counting the number of incorrect choices. The behaviors measured are statistically analyzed using standard statistical tests. (see e.g., Spruijt et al., Brain Res. 527:192-197 (1990)).

Alternatively, a Y-shaped maze may be used (see e.g., McFarland, Pharmacology, Biochemistry and Behavior 32:723-726 (1989); Dellu et al., Neurobiology of Learning and Memory 73:31-48 (2000)). The Y-maze is generally believed to be a test of cognitive ability. The dimensions of each arm of the Y-maze can be, for example, approximately 40 cm×8 cm×20 cm, although other dimensions may be used. Each arm can also have, for example, sixteen equally spaced photobeams to automatically detect movement within the arms. At least two different tests can be performed using such a Y-maze. In a continuous Y-maze paradigm, mice are allowed to explore all three arms of a Y-maze for, e.g., approximately 10 minutes. The animals are continuously tracked using photobeam detection grids, and the data can be used to measure spontaneous alteration and positive bias behavior. Spontaneous alteration refers to the natural tendency of a “normal” animal to visit the least familiar arm of a maze. An alternation is scored when the animal makes two consecutive turns in the same direction, thus representing a sequence of visits to the least recently entered arm of the maze. Position bias determines egocentrically defined responses by measuring the animal's tendency to favor turning in one direction over another. Therefore, the test can detect differences in an animal's ability to navigate on the basis of allocentric or egocentric mechanisms. The two-trial Y-maze memory test measures response to novelty and spatial memory based on a free-choice exploration paradigm. During the first trial (acquisition), the animals are allowed to freely visit two arms of the Y-maze for, e.g., approximately 15 minutes. The third arm is blocked off during this trial. The second trial (retrieval) is performed after an intertrial interval of, e.g., approximately 2 hours. During the retrieval trial, the blocked arm is opened and the animal is allowed access to all three arms for, e.g., approximately 5 minutes. Data are collected during the retrieval trial and analyzed for the number and duration of visits to each arm. Because the three arms of the maze are virtually identical, discrimination between novelty and familiarity is dependent on “environmental” spatial cues around the room relative to the position of each arm. Changes in arm entry and duration of time spent in the novel arm in a transgenic animal model may be indicative of a role of that gene in mediating novelty and recognition processes.

The passive avoidance or shuttle box test generally involves exposure to two or more environments, one of which is noxious, providing a choice to be learned by the animal. Behavioral measures include, for example, response latency, number of correct responses, and consistency of response. (see e.g., Ader et al., Psychon. Sci. 26:125-128 (1972); Holson, Phys. Behav. 37:221-230 (1986)). Alternatively, a zero maze can be used. In a zero-maze, the animals can, for example, be placed in a closed quadrant of an elevated annular platform having, e.g., 2 open and 2 closed quadrants, and are allowed to explore for approximately 5 minutes. This paradigm exploits an approach-avoidance conflict between normal exploratory activity and an aversion to open spaces in rodents. This test measures anxiety levels and can be used to evaluate the effectiveness of anti-anxiolytic drugs. The time spent in open quadrants versus closed quadrants may be recorded automatically, with, for example, the placement of photobeams at each transition site.

The food avoidance test involves exposure to novel food and objectively measuring, for example, food intake and intake latency. The behaviors measured are statistically analyzed using standard statistical tests. (see e.g., Campbell et al., J. Comp. Physiol. Psychol. 67:15-22 (1969)).

The elevated plus-maze test comprises exposure to a maze, without sides, on a platform, the animal's behavior is objectively measured by counting the number of maze entries and maze learning. The behavior is statistically analyzed using standard statistical tests. (see e.g., Baldwin et al., Brain Res. Bull., 20:603-606 (1988)).

The stimulant-induced hyperactivity test involves injection of stimulant drugs (e.g., amphetamines, cocaine, PCP, and the like), and objectively measuring, for example, motor activity, social interactions, cognitive behavior. The animal's behaviors are statistically analyzed using standard statistical tests. (see e.g., Clarke et al., Psychopharmacology 96:511-520 (1988); Kuczenski et al., J. Neuroscience 11:2703-2712 (1991)).

The self-stimulation test generally comprises providing the mouse with the opportunity to regulate electrical and/or chemical stimuli to its own brain. Behavior is measured by frequency and pattern of self-stimulation. Such behaviors are statistically analyzed using standard statistical tests. (see e.g., Nassif et al., Brain Res., 332:247-257 (1985); Isaac et al., Behav. Neurosci. 103:345-355 (1989)).

The reward test involves shaping a variety of behaviors, e.g., motor, cognitive, and social, measuring, for example, rapidity and reliability of behavioral change, and statistically analyzing the behaviors measured. (see e.g., Jarrard et al., Exp. Brain Res. 61:519-530 (1986)).

The DRL (differential reinforcement to low rates of responding) performance test involves exposure to intermittent reward paradigms and measuring the number of proper responses, e.g., lever pressing. Such behavior is statistically analyzed using standard statistical tests. (see e.g., Sinden et al., Behav. Neurosci. 100:320-329 (1986); Nalwa et al., Behav. Brain Res. 17:73-76 (1985); and Normeman et al, J. Comp. Physiol. Psych. 95:588-602 (1981)).

The spatial learning test involves exposure to a complex novel environment, measuring the rapidity and extent of spatial learning, and statistically analyzing the behaviors measured. (see e.g., Pitsikas et al., Pharm. Bioch. Behav. 38:931-934 (1991); Poucet et al., Brain Res. 37:269-280 (1990); Christie et al., Brain Res. 37:263-268 (1990); and Van Haaren et al., Behav. Neurosci. 102:481-488 (1988)). Alternatively, an open-field test may be used, in which the greater distance traveled for a given amount of time is a measure of the activity level and anxiety of the animal. When the open field is a novel environment, it is believed that an approach-avoidance situation is created, in which the animal is “torn” between the drive to explore and the drive to protect itself. Because the chamber is lighted and has no places to hide other than the corners, it is expected that a “normal” mouse will spend more time in the corners and around the periphery than it will in the center where there is no place to hide. “Normal” mice will, however, venture into the central regions as they explore more and more of the chamber. It can then be extrapolated that especially anxious mice will spend most of their time in the corners, with relatively little or no exploration of the central region, whereas bold (i.e., less anxious) mice will travel a greater distance, showing little preference for the periphery versus the central region.

The visual, somatosensory and auditory neglect tests generally comprise exposure to a sensory stimulus, objectively measuring, for example, orientating responses, and statistically analyzing the behaviors measured. (see e.g., Vargo et al., Exp. Neurol. 102:199-209 (1988)).

The consummatory behavior test generally comprises feeding and drinking, and objectively measuring quantity of consumption. The behavior measured is statistically analyzed using standard statistical tests. (see e.g., Fletcher et al., Psychopharmacol. 102:301-308 (1990); Corda et al, Proc. Natl. Acad. Sci. USA 80:2072-2076 (1983)).

A visual discrimination test can also be used to evaluate the visual processing of an animal. One or two similar objects are placed in an open field and the animal is allowed to explore for about 5-10 minutes. The time spent exploring each object (proximity to, i.e., movement within, e.g., about 3-5 cm of the object is considered exploration of an object) is recorded. The animal is then removed from the open field, and the objects are replaced by a similar object and a novel object. The animal is returned to the open field and the percentage time spent exploring the novel object over the old object is measured (again, over about a 5-10 minute span). “Normal” animals will typically spend a higher percentage of time exploring the novel object rather than the old object. If a delay is imposed between sampling and testing, the memory task becomes more hippocampal-dependent. If no delay is imposed, the task is more based on simple visual discrimination. This test can also be used for olfactory discrimination, in which the objects (simple blocks) can be sprayed or otherwise treated to hold an odor. This test can also be used to determine if the animal can make gustatory discriminations; animals that return to the previously eaten food instead of novel food exhibit gustatory neophobia.

A hot plate analgesia test can be used to evaluate an animal's sensitivity to heat or painful stimuli. For example, a mouse can be placed on an approximately 55° C. hot plate and the mouse's response latency (e.g., time to pick up and lick a hind paw) can be recorded. These responses are not reflexes, but rather “higher” responses requiring cortical involvement. This test may be used to evaluate a nociceptive disorder.

An accelerating rotarod test may be used to measure coordination and balance in mice. Animals can be, for example, placed on a rod that acts like a rotating treadmill (or rolling log). The rotarod can be made to rotate slowly at first and then progressively faster until it reaches a speed of, e.g., approximately 60 rpm. The mice must continually reposition themselves in order to avoid falling off. The animals are may be tested in at least three trials, a minimum of 20 minutes apart. Those mice that are able to stay on the rod the longest are believed to have better coordination and balance.

A metrazol administration test can be used to screen animals for varying susceptibilities to seizures or similar events. For example, a 5 mg/ml solution of metrazol can be infused through the tail vein of a mouse at a rate of, e.g., approximately 0.375 ml/minutes. The infusion will cause all mice to experience seizures, followed by death. Those mice that enter the seizure stage the soonest are believed to be more prone to seizures. Four distinct physiological stages can be recorded: soon after the start of infusion, the mice will exhibit a noticeable “twitch,” followed by a series of seizures, ending in a final tensing of the body known as “tonic extension,” which is followed by death.

Phenotypes

The present invention describes a schizophrenia-like phenotype of a transgenic mouse. As used herein, the schizophrenia phenotype demonstrated by patients is characterized by delusions, hallucinations, severe thought disorganization, grossly disorganized behavior, catatonic behavior caused by treatment with typical antipsychotics, inappropriate response to situation or stimuli, unusual motor behavior, depersonalization, derealization, somatic preoccupations, poverty of speech, flattening of affect, apathy, anhedonia, social withdrawal, attentional impairment, deficit in prepulse inhibition, an increased sensitivity to psychomimetic agents, and/or a increased level of dopamine in the striatum. Animal behavioral assays which model a schizophrenia-like phenotype include, but are not limited to, apomorphine-induced behaviors, such as locomotor activity, climbing and stereotypy behaviors such as sniffing, licking, biting, and gnawing.

Additional behavioral assays that model a schizophrenia-like phenotype of the present invention include increased hyperactivity upon amphetamine stimulation (e.g., increased horizontal locomotor activity and vertical rearings) and a deficit in prepulse inhibition of a startle response.

Antipsychotics

Because the present invention identifies and describes an animal model with schizophrenia-like phenotypes, antipsychotic medications should alleviate some, but not all, of the symptoms associated with schizophrenia. Antipsychotics include, but are not limited to, haloperidol, risperidone, clozapine, chlorpromazine, thioridazine, mesoridazine, fluphenazine, perphenazine, trifluoperazine, thiothixene, loxapine, molindone, pimozide, promazine, chlorprothixene, prochlorperazine, olanzapine, quetiapine, sertindole, ziprasidone, aripiprazole, droperidol, acetophenazine, flupenthixol, methotrimeprazine, and pipotiazine. Typical antipsychotics (e.g., haloperidol, a selective D2 antagonist) are most effective in reversing PPI disruptions resulting from treatment with agents that increase D2 activity in the striatum while atypical antipsychotics (e.g., clozapine, a D2 antagonist with activity at many other receptors) are more effective in reversing PPI disruptions resulting from treatment with glutamate antagonists (Swerdlow et al., Behavioral Pharmacology 11:185-204 (2000); Geyer et al., Psychopharmacology 156:117-154 (2001); Geyer and Ellenbroek, Prog Neuropsychopharmacol Biol Psychiatry 27:1071-1079 (2003)).

GPR88 Involvement in Other Diseases

The present invention further describes other diseases in which the GPR88 receptor may have a role. Such diseases include, but are not limited to, schizophrenia, Parkinson disease, Huntington disease, attention deficit hyperactivity disorder (ADHD), hallucination induced by drug abuse or epilepsy, Tourette's syndrome, restless leg syndrome, obsessive-compulsive disorder (OCD), mania, depression, drug addiction, and hyperphagia.

The present invention further contemplates use of the GPR88 receptor gene sequence to produce GPR88 receptor gene products. GPR88 receptor gene products may include proteins that represent functionally equivalent gene products. Such an equivalent gene product may contain deletions, additions or substitutions of amino acid residues within the amino acid sequence encoded by the gene sequences described herein, but which result in a silent change, thus producing a functionally equivalent GPR88 receptor gene product. Amino acid substitutions may be made on the basis of similarity in polarity, charge, solubility, hydrophobicity, hydrophilicity, and/or the amphipathic nature of the residues involved.

For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Functionally equivalent,” as utilized herein, refers to a protein capable of exhibiting a substantially similar in vivo activity as the endogenous gene products encoded by the GPR88 receptor gene sequences. Alternatively, when utilized as part of an assay, “functionally equivalent” may refer to peptides capable of interacting with other cellular or extracellular molecules in a manner substantially similar to the way in which the corresponding portion of the endogenous gene product would.

Other protein products useful according to the methods of the invention are peptides derived from or based on the GPR88 receptor gene produced by recombinant or synthetic means (derived peptides).

GPR88 receptor gene products may be produced by recombinant DNA technology using techniques well known in the art. Thus, methods for preparing the gene polypeptides and peptides of the invention by expressing nucleic acid encoding gene sequences are described herein. Methods that are well known to those skilled in the art can be used to construct expression vectors containing gene protein coding sequences and appropriate transcriptional/translational control signals. These methods include, for example, in vitro recombinant DNA techniques, synthetic techniques and in vivo recombination/genetic recombination (see e.g., Sambrook et al., 1989, Molecular Cloning: A Laboratory Manual, Second Edition, Cold Spring Harbor Laboratory Press, Cold Spring Harbor, N.Y.; Ausubel et al., 1994, Current Protocols in Molecular Biology, John Wiley & Sons, Inc.). Alternatively, RNA capable of encoding gene protein sequences may be chemically synthesized using, for example, automated synthesizers (see, e.g. Gait, M. J. (ed.), Oligonucleotide Synthesis: A Practical Approach, IRL Press, Oxford (1984)).

A variety of host-expression vector systems may be utilized to express the gene coding sequences of the invention. Such host-expression systems represent vehicles by which the coding sequences of interest may be produced and subsequently purified, but also represent cells that may, when transformed or transfected with the appropriate nucleotide coding sequences, exhibit the gene protein of the invention in situ. These include but are not limited to microorganisms such as bacteria (e.g., E. coli, B. subtilis) transformed with recombinant bacteriophage DNA, plasmid DNA or cosmid DNA expression vectors containing gene protein coding sequences; yeast (e.g. Saccharomyces, Pichia) transformed with recombinant yeast expression vectors containing the gene protein coding sequences; insect cell systems infected with recombinant virus expression vectors (e.g., baculovirus) containing the gene protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing gene protein coding sequences; or mammalian cell systems (e.g. COS, CHO, BHK, 293, 3T3) harboring recombinant expression constructs containing promoters derived from the genome of mammalian cells (e.g., metallothionine promoter) or from mammalian viruses (e.g., the adenovirus late promoter; the vaccinia virus 7.5 K promoter; and the cytomegalovirus (CMV) promoter).

In bacterial systems, a number of expression vectors may be advantageously selected depending upon the use intended for the gene protein being expressed. For example, when a large quantity of such a protein is to be produced, for the generation of antibodies or to screen peptide libraries, for example, vectors that direct the expression of high levels of fusion protein products that are readily purified may be desirable. Such vectors include, but are not limited, to the E. coli expression vector pUR278 (Ruther et al., EMBO J., 2:1791-94 (1983)), in which the gene protein coding sequence may be ligated individually into the vector in frame with the lac Z coding region so that a fusion protein is produced; pIN vectors (Inouye & Inouye, Nucleic Acids Res., 13:3101-09 (1985); Van Heeke et al., J. Biol. Chem., 264:5503-9 (1989)) and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned DEZ receptor gene protein can be released from the GST moiety.

In the present invention, full length cDNA sequences are appended with in-frame Bam HI sites at the amino terminus and EcoRI sites at the carboxyl terminus using standard PCR methodologies (Innis et al. (eds), PCR Protocols: A Guide to Methods and Applications, Academic Press, San Diego (1990)) and ligated into the pGEX-2TK vector (Pharmacia, Uppsala, Sweden). The resulting cDNA construct contains a kinase recognition site at the amino terminus for radioactive labeling and glutathione S-transferase sequences at the carboxyl terminus for affinity purification (Nilsson et al., EMBO J., 4: 1075-80 (1985); Zabeau et al., EMBO J., 1: 1217-24 (1982)).

In an insect system, Autographa californica nuclear polyhedrosis virus (AcNPV) is used as a vector to express foreign genes. The virus grows in Spodoptera frugiperda cells. The gene coding sequence may be cloned individually into non-essential regions (for example the polyhedrin gene) of the virus and placed under control of an AcNPV promoter (for example the polyhedrin promoter). Successful insertion of gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (see e.g., Smith et al., J. Virol. 46: 584-93 (1983); U.S. Pat. No. 4,745,051).

In mammalian host cells, a number of viral-based expression systems may be utilized. In cases where an adenovirus is used as an expression vector, the gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing gene protein in infected hosts. (e.g., Logan et al., Proc. Natl. Acad. Sci. USA, 81:3655-59 (1984)). Specific initiation signals may also be required for efficient translation of inserted gene coding sequences. These signals include the ATG initiation codon and adjacent sequences. In cases where an entire gene, including its own initiation codon and adjacent sequences, is inserted into the appropriate expression vector, no additional translational control signals may be needed. However, in cases where only a portion of the gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter et al., Meth. Enzymol., 153:516-44 (1987)).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, etc.

For long-term, high-yield production of recombinant proteins, stable expression is possible. For example, cell lines that stably express the gene product may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells that stably integrate the plasmid into their chromosomes and grow, to form foci, which in turn can be cloned and expanded into cell lines. This method may advantageously be used to engineer cell lines that express the gene protein. Such engineered cell lines may be particularly useful in screening and evaluation of compounds that affect the endogenous activity of the gene protein.

Timing and/or quantity of expression of the recombinant protein can be controlled using an inducible expression construct. Inducible constructs and systems for inducible expression of recombinant proteins will be well known to those skilled in the art. Examples of such inducible promoters or other gene regulatory elements include, but are not limited to, tetracycline, metallothionine, ecdysone, and other steroid-responsive promoters, rapamycin responsive promoters, and the like (No et al., Proc. Natl. Acad. Sci. USA, 93:3346-51 (1996); Furth et al., Proc. Natl. Acad. Sci. USA, 91:9302-6 (1994)). Additional control elements that can be used include promoters requiring specific transcription factors such as viral, particularly HIV, promoters. A Tet inducible gene expression system, or other inducible expression system, may be used. (Gossen et al., Proc. Natl. Acad. Sci. USA, 89:5547-51 (1992); Gossen et al., Science, 268:1766-69 (1995)). Tet Expression Systems are based on two regulatory elements derived from the tetracycline-resistance operon of the E. coli Tn10 transposon—the tetracycline repressor protein (TetR) and the tetracycline operator sequence (tetO) to which TetR binds. Using such a system, expression of the recombinant protein is placed under the control of the tetO operator sequence and transfected or transformed into a host cell. In the presence of TetR, which is cotransfected into the host cell, expression of the recombinant protein is repressed due to binding of the TetR protein to the tetO regulatory element. High-level, regulated gene expression can then be induced in response to varying concentrations of tetracycline (Tc) or Tc derivatives such as doxycycline (Dox), which compete with tetO elements for binding to TetR. Constructs and materials for tet inducible gene expression are available commercially from CLONTECH Laboratories, Inc., Palo Alto, Calif.

When used as a component in an assay system, the gene protein may be labeled, either directly or indirectly, to facilitate detection of a complex formed between the gene protein and a test substance. Any of a variety of suitable labeling systems may be used including but not limited to radioisotopes such as ¹²⁵I enzyme labeling systems that generate a detectable calorimetric signal or light when exposed to substrate and fluorescent labels. Where recombinant DNA technology is used to produce the gene protein for such assay systems, it may be advantageous to engineer fusion proteins that can facilitate labeling, immobilization and/or detection.

Indirect labeling involves the use of a protein, such as a labeled antibody, which specifically binds to the gene product. Such antibodies include but are not limited to polyclonal, monoclonal, chimeric, single chain, Fab fragments and fragments produced by a Fab expression library.

Production of Antibodies

Described herein are methods for the production of antibodies capable of specifically recognizing one or more epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a GPR88 receptor gene in a biological sample, or, alternatively, as a method for the inhibition of abnormal GPR88 receptor gene activity. Thus, such antibodies may be utilized as part of disease treatment methods, and/or may be used as part of diagnostic techniques whereby patients may be tested for abnormal levels of GPR88 receptor gene proteins, or for the presence of abnormal forms of such proteins.

For the production of antibodies, various host animals may be immunized by injection with the GPR88 receptor gene, its expression product or a portion thereof. Such host animals may include but are not limited to rabbits, mice, rats, goats and chickens, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as the GPR88 receptor gene product, or an antigenic functional derivative thereof. For the production of polyclonal antibodies, host animals such as those described above, may be immunized by injection with gene product supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to the hybridoma technique of Kohler and Milstein, Nature, 256:495-7 (1975); and U.S. Pat. No. 4,376,110), the human B-cell hybridoma technique (Kosbor et al., Immunology Today, 4:72 (1983); Cote et al., Proc. Natl. Acad. Sci. USA, 80:2026-30 (1983)), and the EBV-hybridoma technique (Cole et al., in Monoclonal Antibodies And Cancer Therapy, Alan R. Liss, Inc., New York, pp. 77-96 (1985)). Such antibodies may be of any immunoglobulin class including IgG, IgM, IgE, IgA, IgD and any subclass thereof. The hybridoma producing the mAb of this invention may be cultivated in vitro or in vivo.

In addition, techniques developed for the production of “chimeric antibodies” (Morrison et al., Proc. Natl. Acad. Sci. USA, 81:6851-6855 (1984); Takeda et al., Nature, 314:452-54 (1985)) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, Science 242:423-26 (1988); Huston et al., Proc. Natl. Acad. Sci. USA, 85:5879-83 (1988); and Ward et al., Nature, 334:544-46 (1989)) can be adapted to produce single chain antibodies. Single chain antibodies are typically formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments that can be produced by pepsin digestion of the antibody molecule and the Fab fragments that can be generated by reducing the disulfide bridges of the F(ab′)₂ fragments. Alternatively, Fab expression libraries may be constructed (Huse et al., Science, 246:1275-81 (1989)) to allow rapid and easy identification of monoclonal Fab fragments with the desired specificity.

Screening Methods

The present invention may be employed in a process for screening for agents such as agonists, i.e., agents that bind to and activate GPR88 receptor polypeptides, or antagonists, i.e., inhibit the activity or interaction of GPR88 receptor polypeptides with its ligand. Thus, the GPR88 receptor polypeptide may also be used to assess the binding of small molecule substrates and ligands in, for example, cells, cell-free preparations, chemical libraries, and natural product mixtures as known in the art. Any methods routinely used to identify and screen for agents that can modulate receptors may be used in accordance with the present invention.

The present invention provides methods for identifying and screening for agents that modulate GPR88 receptor expression or function. More particularly, cells that contain and express GPR88 receptor gene sequences may be used to screen for therapeutic agents. Such cells may include non-recombinant monocyte cell lines, such as U937 (ATCC# CRL-1593), THP-1 (ATCC# TIB-202), and P388D1 (ATCC# TIB-63); endothelial cells such as HUVEC's and bovine aortic endothelial cells (BAEC's); as well as generic mammalian cell lines such as CHO cells, HeLa cells, and COS cells (e.g., COS-7 (ATCC# CRL-1651)). Further, such cells may include recombinant, transgenic cell lines. For example, the transgenic mice of the invention may be used to generate cell lines, containing one or more cell types involved in a disease, that can be used as cell culture models for that disorder. While cells, tissues, and primary cultures derived from the transgenic animals of the invention may be utilized, the generation of continuous cell lines is are also possible. For examples of techniques that may be used to derive a continuous cell line from the transgenic animals (see Small et al., Mol. Cell Biol., 5:642-48 (1985)).

GPR88 receptor gene sequences may be introduced into, and overexpressed in, the genome of the cell of interest. In order to overexpress a GPR88 receptor gene sequence, the coding portion of the GPR88 receptor gene sequence may be ligated to a regulatory sequence that is capable of driving gene expression in the cell type of interest. Such regulatory regions will be well known to those of skill in the art, and may be utilized in the absence of undue experimentation. Cells having GPR88 receptor gene expressed may be used, for example, to screen for agents capable of affecting signalling pathways well known to those of skill in the art, and may be utilized in the absence of undue experimentation. GPR88 receptor gene sequences may also be disrupted or underexpressed. Cells having GPR88 receptor gene disruptions or underexpressed GPR88 receptor gene sequences may be used, for example, to screen for agents capable of affecting alternative pathways that compensate for any loss of function attributable to the disruption or underexpression.

In vitro systems may be designed to identify compounds capable of binding the GPR88 receptor gene products. Such compounds may include, but are not limited to, peptides made of D- and/or L-configuration amino acids (m, for example, the form of random peptide libraries; (see e.g., Lam et al., Nature, 354:82-4 (1991)), phosphopeptides (m, for example, the form of random or partially degenerate, directed phosphopeptide libraries; see e.g., Songyang et al., Cell, 72:767-78 (1993)), antibodies, and small organic or inorganic molecules. Compounds identified may be useful, for example, in modulating the activity of GPR88 receptor gene proteins, mutant GPR88 receptor gene proteins; elaborating the biological function of the GPR88 receptor gene protein; or screening for compounds that disrupt normal GPR88 receptor gene interactions or themselves disrupt such interactions.

The principle of the assays used to identify compounds that bind to the GPR88 receptor gene protein involves preparing a reaction mixture of the GPR88 receptor gene protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring the GPR88 receptor gene protein or the test substance onto a solid phase and detecting target protein/test substance complexes anchored on the solid phase at the end of the reaction. For example, the GPR88 receptor gene protein may be anchored onto a solid surface, and the test compound, which is not anchored, may be labeled, either directly or indirectly.

In practice, microtitre plates are conveniently utilized. The anchored component may be immobilized by non-covalent or covalent attachments. Non-covalent attachment may be accomplished simply by coating the solid surface with a solution of the protein and drying. Alternatively, an immobilized antibody, (e.g., a monoclonal antibody, specific for the protein) may be used to anchor the protein to the solid surface. The surfaces may be prepared in advance and stored.

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for GPR88 receptor gene product or the test compound to anchor any complexes formed in solution, and a labeled antibody specific for the other component of the possible complex to detect anchored complexes.

Compounds that are shown to bind to a particular GPR88 receptor gene product through one of the methods described above can be further tested for their ability to elicit a biochemical response from the GPR88 receptor gene protein. Agonists, antagonists and/or inhibitors of the expression product can be identified utilizing assays well known in the art.

Antisense, Ribozymes, and Antibodies

Other agents that may be used as therapeutics include the GPR88 receptor gene, its expression product(s) and functional fragments thereof. Additionally, agents that reduce or inhibit mutant GPR88 receptor gene activity may be used to ameliorate disease symptoms. Such agents include antisense, ribozyme, and triple helix molecules. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the GPR88 receptor gene nucleotide sequence of interest, are may be used.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage. The composition of ribozyme molecules must include one or more sequences complementary to the GPR88 receptor gene mRNA, and must include the well known catalytic sequence responsible for mRNA cleavage. For this sequence, see U.S. Pat. No. 5,093,246, which is incorporated by reference herein in its entirety. As such within the scope of the invention are engineered hammerhead motif ribozyme molecules that specifically and efficiently catalyze endonucleolytic cleavage of RNA sequences encoding GPR88 receptor gene proteins.

Specific ribozyme cleavage sites within any potential RNA target are initially identified by scanning the molecule of interest for ribozyme cleavage sites that include the following sequences, GUA, GUU and GUC. Once identified, short RNA sequences of between 15 and 20 ribonucleotides corresponding to the region of the GPR88 receptor gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

It is possible that the antisense, ribozyme, and/or triple helix molecules described herein may reduce or inhibit the transcription (triple helix) and/or translation (antisense, ribozyme) of mRNA produced by both normal and mutant GPR88 receptor gene alleles. In order to ensure that substantially normal levels of GPR88 receptor gene activity are maintained, nucleic acid molecules that encode and express GPR88 receptor gene polypeptides exhibiting normal activity may be introduced into cells that do not contain sequences susceptible to whatever antisense, ribozyme, or triple helix treatments are being utilized. Co-administer normal GPR88 receptor gene protein into the cell or tissue in order to maintain the requisite level of cellular or tissue GPR88 receptor gene activity is also possible.

Anti-sense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Antibodies that are both specific for GPR88 receptor gene protein, and in particular, mutant gene protein, and interfere with its activity may be used to inhibit mutant GPR88 receptor gene function. Such antibodies may be generated against the proteins themselves or against peptides corresponding to portions of the proteins using standard techniques known in the art and as also described herein. Such antibodies include but are not limited to polyclonal, monoclonal, Fab fragments, single chain antibodies, chimeric antibodies, etc.

In instances where the GPR88 receptor gene protein is intracellular and whole antibodies are used, internalizing antibodies may also be used. However, lipofectin liposomes may be used to deliver the antibody or a fragment of the Fab region that binds to the GPR88 receptor gene epitope into cells. Where fragments of the antibody are used, the smallest inhibitory fragment that binds to the target or expanded target protein binding domain may be used. For example, peptides having an amino acid sequence corresponding to the domain of the variable region of the antibody that binds to the GPR88 receptor gene protein may be used. Such peptides may be synthesized chemically or produced via recombinant DNA technology using methods well known in the art (see e.g., Creighton, Proteins: Structures and Molecular Principles W. H. Freeman, New York 1983; Sambrook et al., 1989, supra). Alternatively, single chain neutralizing antibodies that bind to intracellular GPR88 receptor gene epitopes may also be administered. Such single chain antibodies may be administered, for example, by expressing nucleotide sequences encoding single-chain antibodies within the target cell population by utilizing, for example, techniques such as those described in Marasco et al, Proc. Natl. Acad. Sci. USA, 90:7889-93 (1993).

RNA sequences encoding GPR88 receptor gene protein may be directly administered to a patient exhibiting disease symptoms, at a concentration sufficient to produce a level of GPR88 receptor gene protein such that disease symptoms are ameliorated. Patients may be treated by gene replacement therapy. One or more copies of a normal GPR88 receptor gene, or a portion of the gene that directs the production of a normal GPR88 receptor gene protein with GPR88 receptor gene function, may be inserted into cells using vectors that include, but are not limited to adenovirus, adeno-associated virus, and retrovirus vectors, in addition to other particles that introduce DNA into cells, such as liposomes. Additionally, techniques such as those described above may be utilized for the introduction of normal GPR88 receptor gene sequences into human cells.

Cells, (e.g., autologous cells), containing normal GPR88 receptor gene expressing gene sequences may then be introduced or reintroduced into the patient at positions that allow for the amelioration of disease symptoms.

Pharmaceutical Compositions, Effective Dosages, and Routes of Administration

The identified compounds that reverse the schizophrenia-like phenotypes can be administered to a patient at therapeutically effective doses to treat or ameliorate symptoms of the disease. A therapeutically effective dose refers to that amount of the compound sufficient to result in amelioration of symptoms of the disease.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices may be used. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically acceptable carriers or excipients. Thus, the compounds and their physiologically acceptable salts and solvates may be formulated for administration by inhalation or insufflation (either through the mouth or the nose) or oral, buccal, parenteral, topical, subcutaneous, intraperitoneal, intraveneous, intrapleural, intraoccular, intraarterial, or rectal administration. It is also contemplated that pharmaceutical compositions may be administered with other products that potentiate the activity of the compound and optionally, may include other therapeutic ingredients.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. Oral ingestion is possibly the easiest method of taking any medication. Such a route of administration is generally simple and straightforward and is frequently the least inconvenient or unpleasant route of administration from the patient's point of view. However, this involves passing the material through the stomach, which is a hostile environment for many materials, including proteins and other biologically active compositions. As the acidic, hydrolytic and proteolytic environment of the stomach has evolved efficiently to digest proteinaceous materials into amino acids and oligopeptides for subsequent anabolism, it is hardly surprising that very little or any of a wide variety of biologically active proteinaceous material, if simply taken orally, would survive its passage through the stomach to be taken up by the body in the small intestine. The result, is that many proteinaceous medicaments must be taken in through another method, such as parenterally, often by subcutaneous, intramuscular or intravenous injection.

Pharmaceutical compositions may also include various buffers (e.g., Tris, acetate, phosphate), solubilizers (e.g., TWEEN®, Polysorbate), carriers such as human serum albumin, preservatives (thimerosol, benzyl alcohol) and anti-oxidants such as ascorbic acid in order to stabilize pharmaceutical activity. The stabilizing agent may be a detergent, such as TWEEN® 20, TWEEN® 80, NP-40 or TRITON X®-100. EBP may also be incorporated into particulate preparations of polymeric compounds for controlled delivery to a patient over an extended period of time. A more extensive survey of components in pharmaceutical compositions is found in A. R. Gennaro (ed.), Remington Pharmaceutical Sciences, 18th ed., Mack Publishing, Easton, Pa. (1990).

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Diagnostics

A variety of methods may be employed to diagnose disease conditions associated with the GPR88 receptor gene. Specifically, reagents may be used, for example, for the detection of the presence of GPR88 receptor gene mutations, or the detection of either over or under expression of GPR88 receptor gene mRNA.

According to the diagnostic and prognostic method of the present invention, alteration of the wild type GPR88 receptor gene locus is detected. In addition, the method can be performed by detecting the wild type GPR88 receptor gene locus and confirming the lack of a predisposition or neoplasia. “Alteration of a wild type gene” encompasses all forms of mutations including deletions, insertions and point mutations in the coding and noncoding regions. Deletions may be of the entire gene or only a portion of the gene. Point mutations may result in stop codons, frameshift mutations or amino acid substitutions. Somatic mutations are those that occur only in certain tissues, e.g., in tumor tissue, and are not inherited in the germline. Germline mutations can be found in any of a body's tissues and are inherited. If only a single allele is somatically mutated, an early neoplastic state may be indicated. However, if both alleles are mutated, then a late neoplastic state may be indicated. The finding of gene mutations thus provides both diagnostic and prognostic information. A GPR88 receptor gene allele that is not deleted (e.g., that found on the sister chromosome to a chromosome carrying a GPR88 receptor gene deletion) can be screened for other mutations, such as insertions, small deletions, and point mutations. Mutations found in tumor tissues may be linked to decreased expression of the GPR88 receptor gene product. However, mutations leading to non-functional gene products may also be linked to a cancerous state. Point mutational events may occur in regulatory regions, such as in the promoter of the gene, leading to loss or diminution of expression of the mRNA. Point mutations may also abolish proper RNA processing, leading to loss of expression of the GPR88 receptor gene product, or a decrease in mRNA stability or translation efficiency.

One test available for detecting mutations in a candidate locus is to directly compare genomic target sequences from schizophrenic patients with those from a control population. Alternatively, one could sequence messenger RNA after amplification, e.g., by PCR, thereby eliminating the necessity of determining the exon structure of the candidate gene. Mutations from schizophrenic patients falling outside the coding region of the GPR88 receptor gene can be detected by examining the non-coding regions, such as introns and regulatory sequences near or within the GPR88 receptor gene. An early indication that mutations in noncoding regions are important may come from Northern blot experiments that reveal messenger RNA molecules of abnormal size or abundance in schizophrenic patients as compared to control individuals.

The methods described herein may be performed, for example, by utilizing pre-packaged diagnostic kits comprising at least one specific gene nucleic acid or anti-gene antibody reagent described herein, which may be conveniently used, e.g., in clinical settings, to diagnose patients exhibiting disease symptoms or at risk for developing disease.

Any cell type or tissue, ideally striatal neurons, in which the gene is expressed may be utilized in the diagnostics described below.

DNA or RNA from the cell type or tissue to be analyzed may easily be isolated using procedures that are well known to those in the art. Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, PCR In Situ Hybridization: Protocols and Applications, Raven Press, N.Y. (1992)).

Gene nucleotide sequences, either RNA or DNA, may, for example, be used in hybridization or amplification assays of biological samples to detect disease-related gene structures and expression. Such assays may include, but are not limited to, Southern or Northern analyses, restriction fragment length polymorphism assays, single stranded conformational polymorphism analyses, in situ hybridization assays, and polymerase chain reaction analyses. Such analyses may reveal both quantitative aspects of the expression pattern of the gene, and qualitative aspects of the gene expression and/or gene composition. That is, such aspects may include, for example, point mutations, insertions, deletions, chromosomal rearrangements, and/or activation or inactivation of gene expression.

Diagnostic methods for the detection of gene-specific nucleic acid molecules may involve for example, contacting and incubating nucleic acids, derived from the cell type or tissue being analyzed, with one or more labeled nucleic acid reagents under conditions favorable for the specific annealing of these reagents to their complementary sequences within the nucleic acid molecule of interest. The lengths of these nucleic acid reagents are at least 9 to 30 nucleotides. After incubation, all non-annealed nucleic acids are removed from the nucleic acid: fingerprint molecule hybrid. The presence of nucleic acids from the fingerprint tissue that have hybridized, if any such molecules exist, is then detected. Using such a detection scheme, the nucleic acid from the tissue or cell type of interest may be immobilized, for example, to a solid support such as a membrane, or a plastic surface such as that on a microtitre plate or polystyrene beads. In this case, after incubation, non-annealed, labeled nucleic acid reagents are easily removed. Detection of the remaining, annealed, labeled nucleic acid reagents is accomplished using standard techniques well-known to those in the art.

Alternative diagnostic methods for the detection of gene-specific nucleic acid molecules may involve their amplification, e.g., by PCR (see U.S. Pat. No. 4,683,202), ligase chain reaction (Barany, Proc. Natl. Acad. Sci. USA, 88:189-93 (1991)), self sustained sequence replication (Guatelli et al., Proc. Natl. Acad. Sci. USA, 87:1874-78 (1990)), transcriptional amplification system (Kwoh et al., Proc. Natl. Acad. Sci. USA, 86:1173-77 (1989)), Q-Beta Replicase (Lizardi et al., Bio/Technology, 6:1197 (1988)), or any other nucleic acid amplification method, followed by the detection of the amplified molecules using techniques well known to those of skill in the art. These detection schemes are especially useful for the detection of nucleic acid molecules if such molecules are present in very low numbers.

A cDNA molecule may also be obtained from an RNA molecule of interest (e.g., by reverse transcription of the RNA molecule into cDNA). Cell types or tissues from which such RNA may be isolated include any tissue in which wild type fingerprint gene is known to be expressed, including, but not limited, to platelets, neutrophils and lymphocytes. A sequence within the cDNA is then used as the template for a nucleic acid amplification reaction, such as a PCR amplification reaction, or the like. The nucleic acid reagents used as synthesis initiation reagents (primers) in the reverse transcription and nucleic acid amplification steps of this method may be chosen from among the gene nucleic acid reagents described herein. The lengths of such nucleic acid reagents are at least 15-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Antibodies directed against the GPR88 receptor polypeptide may also be used as disease diagnostics and prognostics. Such diagnostic methods, may be used to detect abnormalities in the level of gene protein expression, or abnormalities in the structure and/or tissue, cellular, or subcellular location of fingerprint gene protein. Structural differences may include, for example, differences in the size, electronegativity, or antigenicity of the mutant fingerprint gene protein relative to the normal fingerprint gene protein.

Protein from the tissue or cell type to be analyzed may easily be detected or isolated using techniques that are well known to those of skill in the art, including but not limited to Western blot analysis. For a detailed explanation of methods for carrying out Western blot analysis, see Sambrook et al. (1989) supra, at Chapter 18. The protein detection and isolation methods employed herein may also be such as those described in Harlow and Lane, Antibodies: A Laboratory Manual Cold Spring Harbor Laboratory Press, Cold Spring Harbor, N.Y. (1988).

Diagnostic methods for the detection of wild type or mutant gene peptide molecules may involve, for example, immunoassays wherein fingerprint gene peptides are detected by their interaction with an anti-fingerprint gene-specific peptide antibody.

For example, antibodies, or fragments of antibodies useful in the present invention may be used to quantitatively or qualitatively detect the presence of wild type or mutant gene peptides. This can be accomplished, for example, by immunofluorescence techniques employing a fluorescently labeled antibody (see below) coupled with light microscopic, flow cytometric, or fluorimetric detection. Such techniques are useful if the fingerprint gene peptides are expressed on the cell surface.

The antibodies (or fragments thereof) useful in the present invention may, additionally, be employed histologically, as in immunofluorescence or immunoelectron microscopy, for in situ detection of fingerprint gene peptides. In situ detection may be accomplished by removing a histological specimen from a patient, and applying thereto a labeled antibody of the present invention. The antibody (or fragment) is applied by overlaying the labeled antibody (or fragment) onto a biological sample. Through the use of such a procedure, it is possible to determine not only the presence of the fingerprint gene peptides, but also their distribution in the examined tissue. Using the present invention, those of ordinary skill will readily perceive that any of a wide variety of histological methods (such as staining procedures) can be modified in order to achieve such in situ detection.

Immunoassays for wild type, mutant, or expanded fingerprint gene peptides typically comprise incubating a biological sample, such as a biological fluid, a tissue extract, freshly harvested cells, or cells that have been incubated in tissue culture, in the presence of a detectably labeled antibody capable of identifying fingerprint gene peptides, and detecting the bound antibody by any of a number of techniques well known in the art.

The biological sample may be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled gene-specific antibody. The solid phase support may then be washed with the buffer a second time to remove unbound antibody. The amount of bound label on solid support may then be detected by conventional means.

The terms “solid phase support or carrier” are intended to encompass any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Supports may include polystyrene beads. Those skilled in the art will know many other suitable carriers for binding antibody or antigen, or will be able to ascertain the same by use of routine experimentation.

The binding activity of a given lot of anti-wild type or mutant fingerprint gene peptide antibody may be determined according to well known methods. Those skilled in the art will be able to determine operative and optimal assay conditions for each determination by employing routine experimentation.

One of the ways in which the gene peptide-specific antibody can be detectably labeled is by linking the same to an enzyme and using it in an enzyme immunoassay (EIA) (Voller, Ric. Clin. Lab, 8:289-98 (1978) [“The Enzyme Linked Immunosorbent Assay (ELISA),” Diagnostic Horizons 2:1-7, 1978, Microbiological Associates Quarterly Publication, Walkersville, Md.]; Voller et al., J. Clin. Pathol., 31:507-20 (1978); Butler, Meth. Enzymol., 73:482-523 (1981); Maggio (ed.), Enzyme Immunoassay, CRC Press, Boca Raton, Fla. (1980); Ishikawa et al., (eds.) Enzyme Immunoassay, Igaku-Shoin, Tokyo (1981)). The enzyme that is bound to the antibody will react with an appropriate substrate (e.g., a chromogenic substrate) in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type, mutant, or expanded peptides through the use of a radioimmunoassay (RIA) (see e.g., Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Throughout this application, various publications, patents and published patent applications are referred to by an identifying citation. The disclosures of these publications, patents and published patent specifications referenced in this application are hereby incorporated by reference into the present disclosure to more fully describe the state of the art to which this invention pertains.

The following examples are intended only to illustrate the present invention and should in no way be construed as limiting the subject invention.

### EXAMPLES

### Example 1

### Preparation of GPR88 Knockout Mice

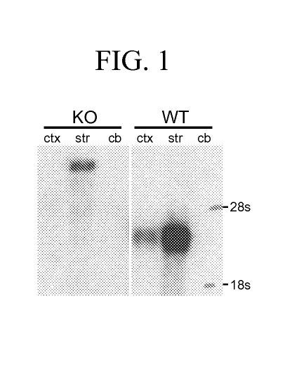

GPR88 knockout mice were prepared by homozygous breeding within KO and WT lines originating from an F1 cross of 129 and C57BL/6 supplied by Lexicon Genetics Incorporated (The Woodlands, Tex.). In brief, gene trapping and gene targeting technologies were used to generate knockout mice by altering the gpr88 locus in embryonic stem cells, which were thereafter cloned and used to generate mice with the altered gpr88 gene. Additional details regarding the techniques for preparing the GPR88 knockout mice can be found in U.S. Pat. Nos. 5,464,764; 5,487,992; 5,627,059; 5,631,153; and 5,789,215, each of which is incorporated by reference in its entirety herein. FIG. 1 shows that GPR88 KO mice do not produce gpr88 transcripts, i.e., that the genetic knockout is a gpr88 null mutation.

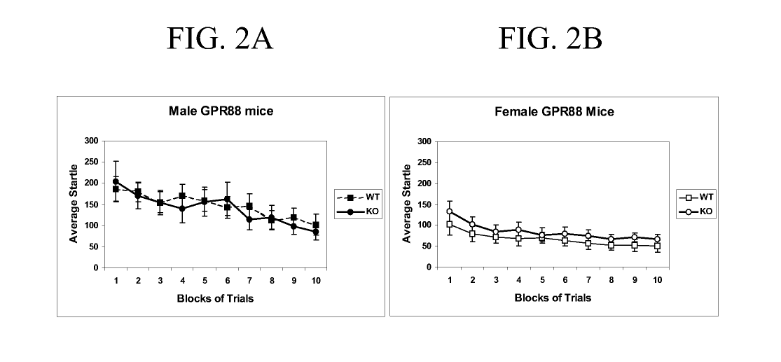

For the functional studies described in the examples herein below, offspring from homozygous breeding pairs for the GPR88 knockout and wild type mice (129Lex mice obtained from Lexicon served as the wild type line) were used for the data shown in FIGS. 2A-2B, 3A-3B, 4A-4B, 5A-5B, 6, 7A-7D, 8A-8D, 9A-9B, and 13A-13B. Offspring from heterozygous breeding pairs, with the heterozygous mice initially produced from pairings of the GPR88 knockout and wild type 129Lex mice already producing homozygous offspring, were used for the data shown in FIGS. 1A-10B, 11A-11B, and 12A-12C. Additional studies confirmed that there is no detectable difference in the phenotype of the GPR88 knockout mice produced by homozygous and heterozygous breeding.

### Example 2

### Startle Habituation Behavioral Assay

To detect potential phenotypic deficits in the GPR88 knockout mice, they were first evaluated for startle habituation. In brief, mice were tested using the SR-LAB (San Diego Instruments, San Diego, Calif.) system. Each system consists of a ventilated sound attenuated chamber containing a stabilimeter consisting of a Plexiglas cylinder (12.5 cm length, 3.5 cm diameter) mounted on a Plexiglas frame. Acoustic stimuli were delivered through a speaker mounted in the enclosure ceiling, that was 27 cm above the cylinder. Startle responses were transduced by a piezoelectric accelerometer mounted beneath the frame. Output signals were digitized and rectified as consecutive 1-millisecond (msec) readings (a total of 65 were recorded, and startle response was expressed as an averaged response across these samples) on a computer using the San Diego Instrument software. Mice were placed individually in the chamber for a 5-minute acclimation period at a background noise level of 64 dB.

After acclimation in the chamber, the mice were exposed to 100 consecutive 110 dB (4 msec duration) acoustic bursts. These acoustic bursts were separated by a 15 second inter-trial interval. Startle data were expressed in 10 blocks of 10 trials each and analyzed with a repeated measure of analysis of variance (ANOVA). If necessary, a post-hoc analysis on the least squares means followed each block of 10 trials. Males and females were analyzed separately.

FIG. 2A shows there was no significant genotype effect (F(1, 18)=0.04, p>0.05) on startle habituation with the male mice. In addition, there was no significant genotype/trial block interaction (F(9, 162)=0.93, p>0.05). A significant decrease in startle response was measured across the trial blocks in both the male KO and WT mice (F(9, 162)=10.74, p<0.05).

The profile was the same for the female mice. See FIG. 2B. No significant genotype effect (F(1, 18)=0.41, p>0.05) nor a significant genotype/trial block interaction (F(9, 162)=0.33, p>0.05) was observed between the female KO and WT mice. However, similar to the male KO and WT mice, there was a significant decrease in startle across the trial blocks in both female KO and WT mice (F(9, 162)=10.84, p<0.05). This indicates that the GPR88 deletion did not have a significant effect on the KO mice's ability to acclimate to a constant startle stimulus as compared to the wild type mice.

### Example 3

### Startle Sensitivity Assay

Next, a startle sensitivity test was performed on the GPR88 knockout and wild type mice. These mice were tested for startle sensitivity response using the SR-LAB as described above in Example 1.

Startle sensitivity was measured at least one day after assessing startle habituation. One of eight startle intensities ranging from 90 to 125 db (in 5 dB increments) was used in assessing startle habituation. Specifically, each mouse was given a 5-minute acclimation period. Then, each mouse was presented with one of the eight different startle intensities over a 40 trial period wherein each trial was separated by a 15 second inter-trial interval. Each startle intensity was presented 5 times over the course of the session, with the order of presentation pseudo-randomized to balance distribution of the various trial types. These 5 trials were then averaged for each mouse to give a individual response at each startle intensity. The response to each startle intensity was analyzed by repeated-measures ANOVA. Again, if necessary, a post-hoc analysis was performed on the least squares means. The males and females were analyzed separately.

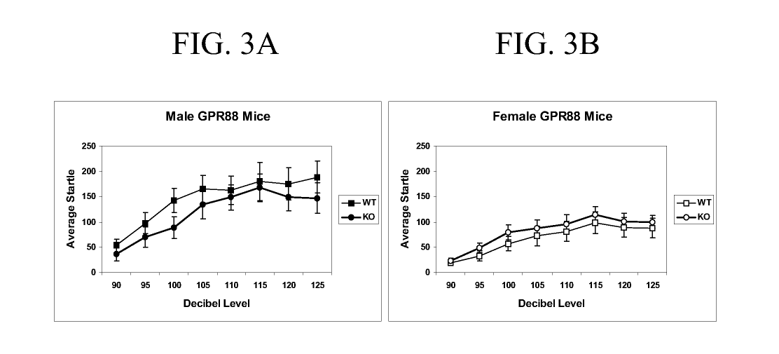

There was no significant genotype effect on startle sensitivity (F(1, 18)=0.72, p>0.05) with the male mice (FIG. 3A). In addition, there was no significant genotype/decibel level interaction (F(7, 126)=0.62, p>0.05). A significant increase in startle was measured as the decibel levels increased in both the male KO and WT mice (F(7, 126)=26.59, p<0.05).

The profile was the same for the female mice (FIG. 3B). No significant genotype effect (F(1, 18)=0.50, p>0.05) nor a significant genotype/decibel level interaction was observed between the female KO and WT mice (F(7, 126)=0.22, p>0.05). Like the male KO and WT mice, however, there was a significant increase in startle as the decibel levels increased (F(7, 126)=29.93, p<0.05) in both genotypes. These results indicate that the GPR88 KO mice had a similar startle response as the WT mice at each of the decibel levels tested.

### Example 4

### Prepulse Inhibition of Startle Response

To study a possible deficit in auditory gating as seen in schizophrenic patients, a prepulse inhibition of startle response assay (PPI) was conducted following the startle sensitivity test. Startle response of the mice was again measured using the SR-LAB discussed above.

Prepulse inhibition was measured immediately following the startle sensitivity test. Five different trial tests were used in studying prepulse inhibition and were performed in a pseudorandom order. These trial tests included (1) a no stimulus trial (64 dB background noise to determine basal activity), a pulse alone trial (118 dB, 40 ms duration), and three 20 ms prepulses at either 69, 76, or 84 dB intensities paired (100 ms, onset to onset) with the 118 dB pulse. Following a 5-minute acclimation period, each mouse was presented with 50 trials with an average inter-trial interval of 15 seconds using the five different trial tests described above. Percentage prepulse inhibition was calculated as 100 minus the ratio of the average startle response observed at each prepulse+pulse trial relative to the pulse alone stimulus, times 100. The percentage prepulse at across dB levels was analyzed by repeated-measures ANOVA. If necessary, a post-hoc analysis followed the ANOVA calculations. Males and females were analyzed separately. Ten wild type male mice, 8 female wild type female mice, 11 male GPR88 KO mice, and 11 female GPR88 KO mice were used in the prepulse inhibition tests.

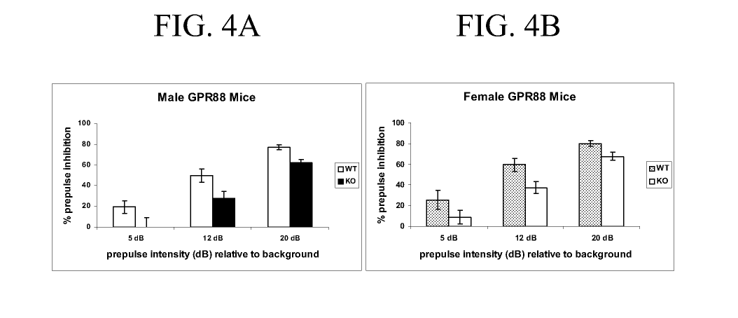

The male mice exhibited a significant genotype effect (F(1, 19)=6.79, p<0.05) with the KO male mice showing a lower prepulse inhibition (PPI) than the WT male mice (FIG. 4A). There was also a significant increase in PPI across the prepulse decibel levels (F(2, 38)=124.71, p<0.05) in both the WT and KO male mice. Although there was no significant genotype/prepulse dB level interaction (F(2, 38)=0.41, p>0.05), the KO mice did have lower PPI across the prepulse dB levels upon post-hoc analysis (p=0.03, p=0.01 and p=0.08 for 5, 12, and 20 dB, respectively).

The profile was similar for the female mice (FIG. 4B), with a significant genotype effect (F(1, 17)=6.98, p<0.05) with the KO mice showing lower PPI than the WT. There was also a significant increase in PPI across the prepulse decibel levels (F(2, 34)=71.26, p<0.05) in both the WT and KO mice. Although there was no significant genotype/prepulse dB level interaction (F(2, 34)=0.53, p>0.05), the KO mice did have lower PPI at the prepulse dB levels upon post-hoc analysis (p=0.055, p=0.01 and p=0.15 for 5, 12, and 20 dB, respectively). The PPI tests demonstrate that the GPR88 KO mice exhibit a deficit in auditory gating. As demonstrated in the startle habituation and startle sensitivity test in Examples 2 and 3, this deficit in auditory gating was not due to genotypic differences in basal startle reactivity.

### Example 5

### Antipsychotic Effects on Prepulse Inhibition of Startle Response

To investigate the efficacy of current antipsychotic medications in the PPI assay using GPR88 null mice, the typical antipsychotic haloperidol and the atypical antipsychotic risperdone were used to dose the GPR88 KO mice before a PPI testing session as described above in Example 4. Specifically, the mice were either dosed with vehicle, haloperidol (0.54 mg/kg) or risperidone (0.54 mg/kg) intraperitoneally (i.p.) 30 minutes before testing in a session as described above. Haloperidol (Sigma, St. Louis, Mo.) was solubilized in 0.25% TWEEN® 80 with 15 minutes of sonication. Risperidone (Toronto Research Chemical, Inc., Ontario, Canada) was solubilized in 0.25% TWEEN® 80. Dosing volumes were 10 ml/kg.

The PPI was averaged across the 3 dB levels and analyzed by a two-way ANOVA with genotype and treatment as the factors followed by a post hoc analysis of least square means. Males and females were again analyzed separately.

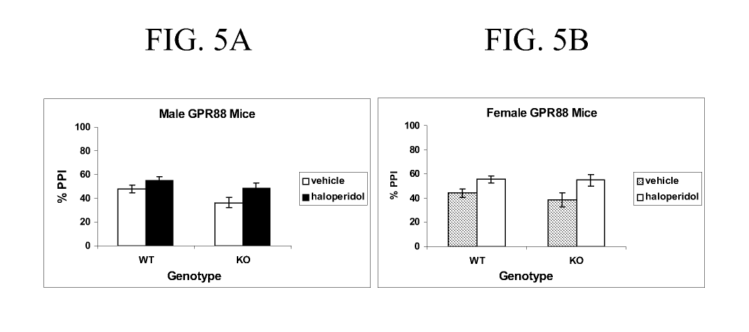

As described in Example 4, a significant genotypic effect on the percentage prepulse inhibition was observed between wild type and GPR88 KO male mice (F(1, 66)=5.08, p<0.05) (FIG. 5A). Haloperidol treatment significantly improved the PPI (F(1, 66)=5.69, p<0.05) regardless of genotype. In terms of a genotype/treatment interaction however (i.e., the difference between percentage PPI of KO and WT mice treated with vehicle vs. the difference between percentage PPI of KO and WT mice treated with haloperidol), haloperidol only improved PPI in the KO mice (p<0.05) and not in the WT mice (p>0.05) (See FIG. 5A; (F(1, 66)=0.38, p>0.05)).

In the female mice, there was no genotype effect between KO mice and WT mice, even though the KO mice trended toward lower PPI than the WT mice (F(1, 68)=1.68, p>0.05). There was a significant increase in PPI with haloperidol treatment (F(1, 68)=10.80, p<0.05) regardless of genotype. However, no significant genotype/treatment interaction (F(1, 68)=0.30, p>0.05) was observed even though haloperidol treated KO mice exhibited an improvement of PPI (p<0.05), while the WT mice (p>0.05) did not exhibit significant improvement upon haloperidol treatment (FIG. 5B).

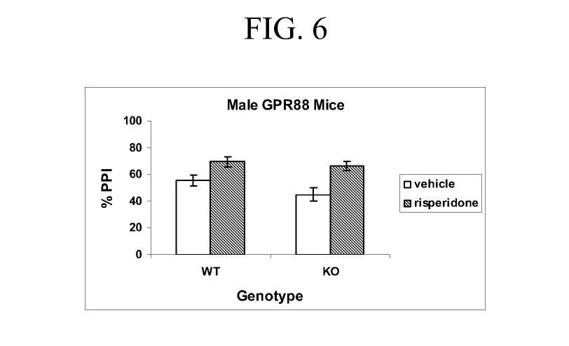

The antipsychotic agent risperidone was also studied using the GPR88 KO mice in PPI assays. Only male mice were studied regarding the effects of risperidone. There was no significant genotype effect observed between KO and WT male mice (F(1, 36)=2.92, p=0.09) although KO mice trended toward lower PPI than the WT mice (FIG. 6). A significant treatment effect of risperidone was observed in both the GPR88 KO (p<0.001) and WT male mice (p<0.02) increasing PPI over untreated. Overall genotypic/treatment interaction was not observed (F(1, 36)=0.84, p>0.05) (FIG. 6).

These results indicate that GPR88 KO mice demonstrate a clear deficit in PPI, but this deficit in PPI is normalized upon treatment with the antipsychotic agents haloperidol and risperidone. Thus, the GPR88 KO mice are an effective tool in studying the efficacy of current antipsychotic medications in the PPI assay.

### Example 6

### Apomorphine-Induced Climbing and Stereotypy

The apomorphine-induced climbing and stereotypy assay is also used extensively to model one of the hypothesized neuropathological states in schizophrenia, the hyperdopaminergia in the ventral striatum. Since GPR88 has striatal-specific expression, the GPR88 KO mouse was tested to see whether it was more sensitive in the apomorphine-induced climbing and stereotypy assay.

The GPR88 KO and WT mice were acclimated to a wire cage (cylindrical ½″×½″ mesh with the dimensions of 4½″ d×5½″ h) for at least 2 hours before testing to allow proper habituation. To quantify the behavioral effects of apomorphine, animals were scored for their ability to demonstrate climbing behavior (0=no climbing, all four feet on the bottom of the cage, 1=two feet up on wire cage, 2=all four feet up on wire cage) and stereotypy behavior, e.g., sniffing, licking, gnawing, etc. (+ present, − absent). Readings were repeated every 5 minutes during a 30-minute test session.

The behavioral assessment was conducted over three conditions in succession: (1) an evaluation of the response to a subcutaneous vehicle (0.25% TWEEN® 80) injection to establish a baseline; (2) followed by an evaluation of the response to a dose of 0.3 mg/kg subcutaneous apomorphine injection; (3) followed by an evaluation of the response to 1 mg/kg subcutaneous apomorphine injection. Behavioral observation began 5 minutes after each injection. Vehicle was given at T0 minutes with observational assessment from T5-T35 minutes and habituation followed from T35-T65 minutes.

At T65, the 0.3 mg/kg dose of apomorphine was administered. Behavioral observation for the 0.3 mg/kg dose of apomorphine was from T70-T100, followed by another habituation from T100-T130 minutes.

At T130 min, the 1.0 mg/kg dose of apomorphine was administered and then the mice were scored from T135-T165 min. For each subject, the climbing scores were totaled for each observational period (maximum=12 per animal). Stereotypy scores were also summed up (maximum=6 per animal). Statistical significance was determined by repeated-measures ANOVA with genotype and treatment as factors followed by post-hoc analysis of least squares means. Males and females were analyzed separately.

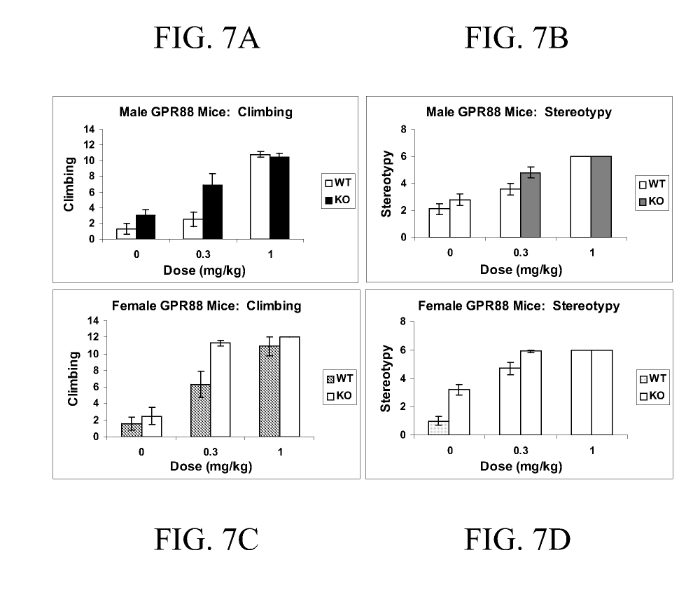

In the male GRP88 KO and WT mice, the overall effect of genotype on apomorphine-induced climbing trended toward significant (F(1, 18)=3.77, p=0.07) with the male GPR88 KO mice trending toward more climbing than the WT based upon post-hoc analysis (p=0.07) (FIG. 7A). A significant dose effect (F(2, 36)=86.33, p<0.05) was observed in the climbing scores for both the male GPR88 KO and WT mice, with climbing increasing with the dose of apomorphine.

A significant interaction was observed between genotype and treatment (F(2, 36)=6.65, p<0.05) with the male KO mice showing higher climbing scores at the 0.3 mg/kg dose than the WT mice. Climbing observed at 1 mg/kg of apomorphine was equivalent between the KO and WT mice.

Similar outcomes were observed with the apomorphine-induced stereotypy measurement in the male mice (FIG. 7B). The overall genotype effect again trended toward significance (F(1, 18)=3.37, p=0.08) with the male GPR88 KO mice showing slightly more stereotypy than the male WT mice. A significant dose effect (F(2, 36)=80.07, p<0.05) was observed in the stereotypy scores for both the male GPR88 KO and WT mice with 1 mg/kg apomorphine producing maximal stereotypy.

Although there was not a significant genotype/dose interaction for stereotypy (F(2, 38)=2.31, p>0.05), the male GPR88 KO mice did show more stereotypy at 0.3 mg/kg apomorphine than did the male WT mice after a post-hoc analysis (p<0.05). The stereotypy observed at 1 mg/kg of apomorphine was equivalent between the KO and WT mice.

A significant genotype effect was observed in the female mice with GPR88 KO female mice showing more climbing than the WT female mice (F(1, 18)=7.91, p<0.05) (FIG. 7C). A significant dose effect (F(2, 36)=54.16, p<0.05) was also observed in both the GPR88 KO and WT females with 1 mg/kg of apomorphine showing the most climbing.

The genotype/dose interaction (F(2, 36)=3.08, p=0.0582) was nearly significant with the female GPR 88 KO mice showing higher climbing scores at the 0.3 mg/kg dose following post-hoc analysis (p<0.05). Again, climbing observed at 1 mg/kg of apomorphine was equivalent between the KO and WT mice.

For apomorphine-induced stereotypy in the female mice, a significant genotype effect (F(1, 18)=23.12, p<0.05) was observed with the female GPR88 KO mice showing more stereotypy than the WT female mice (FIG. 7D). The female GPR88 KO and WT female mice showed a significant dose effect (F(2, 36)=131.90, p<0.05) with 1 mg/kg of apomorphine producing the most stereotypy.

In contrast to the male apomorphine-induced stereotypy, a significant genotype/dose interaction (F(2, 36)=9.25, p<0.05) was observed due to the female KO mice showing more stereotypy following both vehicle treatment and at the 0.3 mg/kg dose of apomorphine.

Overall, the GPR88 KO mice exhibited a greater sensitivity to apomorphine induced climbing and stereotypy than the wild type mice. For both female and male GPR88 KO mice, this sensitivity of the genotypic dose effect was observed at the 0.3 mg/kg dose.

Interestingly, the climbing behavior was more sensitive to apomorphine than the stereotypy behavior, indicating a differential response between the mesolimbic and nigrostriatal system. Specifically, with climbing scores the GPR88 KO mice exhibited an average 227% increase in climbing scores over the wild type mice. With stereotypy scores, the GPR88 KO mice exhibited an average of 129% increase over the wild type mice. Overall, the GPR88 KO mice sensitivity to apomorphine-induced behavior is indicative of a schizophrenic-like phenotype.

### Example 7

### Antipsychotic Block of Apomorphine-Induced Climbing and Stereotypy

The apomorphine-induced climbing and stereotypy assays can also be used to assess the clinical efficacy of antipsychotic agents and help to determine whether antipsychotic agents have typical or atypical profiles (Geyer and Ellenbrook, Prog. Neuropsychopharmacol. Biol. Psychiatry 27:1071-1079 (2003). As described in Example 6, systemic administration of apomorphine activates both the mesolimbic and nigrostriatal pathways resulting in climbing and stereotypy behavior, respectively (Costall et al., Eur. J. Pharmacol. 50:39-50 (1978), Costall et al., Br. J. Pharmacol. 63:381-382 (1978), Costall et al., Br. J. Pharmacol. 68:175-176 (1980), and Hirsch et al., Psychopharmacology 156:117-154 (1987), and the differentiations of antipsychotics into typical and atypical profiles can be made by the degree of separation in doses required to block climbing vs. stereotypy (Geyer and Ellenbrook, 2003). Typical antipsychotics block climbing and stereotypy at the same dose, while atypical antipsychotics block climbing at doses lower than that required to block stereotypy. The antipsychotic agent haloperidol was chosen to study the blocking of apomorphine-induced behaviors.

After habituation, the mice were dosed via the intraperitoneal route with either vehicle or haloperidol. Separate groups of animals were tested for each dose of haloperidol (0.3, 0.54, or 1.0 mg/kg).

After a 30-minute period post-treatment with haloperidol, the mice were given apomorphine (0.54 mg/kg). Observation and scoring for climbing and stereotypy were performed as described in Example 6. The scores for climbing and stereotypy were analyzed by a two-way ANOVA with genotype and haloperidol dose as the factors followed by a post-hoc analysis of least squares means. Males and females were analyzed separately. A minimal effective dose of haloperidol (MED) was defined as the lowest dose that resulted in a significant blockade of apomorphine-induced climbing or stereotypy. For graphical representation, the data was normalized to the amount of climbing and stereotypy shown by the vehicle treated control group. Specifically, these data were presented as the percentage of climbing and percentage of stereotypy shown by control mice (no haloperidol treatment) (FIGS. 7A-7D).

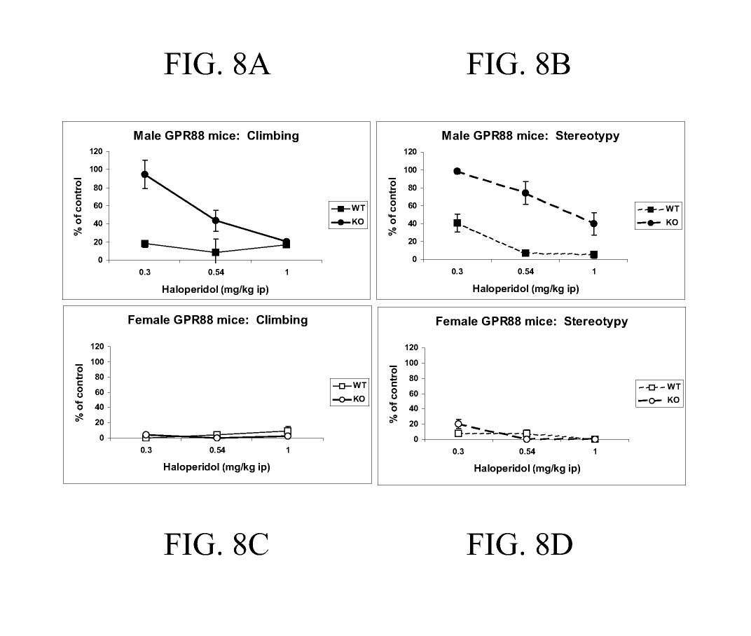

The results indicate that when haloperidol was used to block apomorphine-induced climbing in the male mice (FIG. 8A), there was a significant genotype effect (F(1, 72)=9.93, p<0.05) with the GPR88 KO mice exhibiting less block of climbing than the WT mice. A significant haloperidol dose effect (F(3, 72)=18.96, p<0.05) on both the wild type and GPR88 KO male mice occurred with higher doses of haloperidol producing the greatest block of climbing. In addition, a significant genotype/dose interaction was observed (F(3, 72)=4.43, p<0.05) due to the GPR88 KO male mice showing less sensitivity to haloperidol's ability to block apomorphine-induced climbing. The MED for haloperidol in GPR88 KO male mice was 0.54 mg/kg, while the WT male mice MED was less than or equal to 0.3 mg/kg.

The same pattern was seen for the effect of haloperidol on apomorphine-induced stereotypy measure in the male mice (FIG. 8B), where there was a significant genotype effect (F(1, 72)=60.53, p<0.05) with the GPR88 KO mice showing less block of stereotypy than the WT mice. A significant dose effect in both the WT and GPR88 KO male mice occurred (F(3, 72)=36.75, p<0.05) with the higher doses of haloperidol producing the greatest block of stereotypy. A significant genotype/dose interaction (F(3, 72)=6.29, p<0.05) was also observed due to the GPR88 KO mice showing less sensitivity to haloperidol's ability to block apomorphine-induced stereotypy. The MED in male GPR88 KO mice was 0.54 mg/kg of haloperidol, while the WT male mice's MED was less than or equal to 0.3 mg/kg of haloperidol.

In contrast, the female mice did not exhibit a significant genotype effect (F(1, 71)=3.34, p>0.05), but there was a significant dose effect (F(3, 71)=38.47, p<0.05) with haloperidol resulting in the block of climbing relative to vehicle (FIG. 8C). In fact, both GPR88 KO and WT female mice showed full block of climbing at the 0.3 mg/kg dose of haloperidol. So lower doses of haloperidol would need to be tested to evaluate the sensitivity to haloperidol's block of apomorphine-induced climbing in female mice. There was also a significant genotype/dose interaction (F(3, 71)=3.54, p<0.05) resulting from the greater level of climbing in the KO mice treated with vehicle relative to the WT vehicle treated mice.

The effect of haloperidol on apomorphine-induced stereotypy was similar to that with climbing. In the female mice, there was not a significant genotype effect (F(1, 71)=3.50, p>0.05) (FIG. 8D), but there was a significant dose effect (F(3, 71)=276.17, p<0.05) with all doses of haloperidol blocking stereotypy relative to vehicle (FIG. 8D). A significant genotype/dose interaction was observed (F(3, 71)=3.53, p<0.05), with the KO mice showing more stereotypy with vehicle and 0.3 mg/kg treatment compared to the WT mice. As with climbing, both the GPR88 KO and WT female mice showed full block of stereotypy at 0.3 mg/kg of haloperidol indicating that a lower dose range of haloperidol must be evaluated to determine the sensitivity to haloperidol's block of apomorphine-induced stereotypy in females.

The results demonstrate that the schizophrenia-like phenotype seen in the apomorphine-induced climbing and stereotypy assay was effectively reversed by treatment with the antipsychotic haloperidol in both the GPR88 KO and WT male and female mice. The male KO mice, however, had reduced sensitivity to the effects of haloperidol as seen in the difference between the minimal effective dose observed in the GPR88 KO (0.54 mg/kg) compared with the WT (<0.3 mg/kg) male mice. Consistent with the differential sensitivity between the mesolimbic and nigrostriatal pathways to apomorphine for the induction of climbing and stereotypy, there was a differential response to haloperidol's effects on climbing vs. stereotypy in the male GPR88 KO and WT mice. In the male WT mice, the doses producing full block of climbing (0.54 and 1.0 mg/kg) also produced a full block of stereotypy. In contrast, the male GPR88 KO mice required a 1.0 mg/kg dose of haloperidol to produce a full block of climbing, while this same dosage only produced a 50% block of stereotypy.

Both the WT and GPR88 KO female mice exhibited more sensitivity to the selected dose of haloperidol. Testing at lower dosages will be required to determine if the female GPR88 KO mice are also less sensitive to the antipsychotic effects of haloperidol.

### Example 8

### Sensitivity to Haloperidol-Induced Catalepsy

GPR88 KO mice were also analyzed for their response to haloperidol in a catalepsy assay due their resistance to haloperidol blockage of climbing and stereotypy. The catalepsy assay is a predicative assay for extrapyramidal side effect (EPS) liability.

For induction of catalepsy, 1 mg/kg of haloperidol was administered intraperitoneally 30 minutes prior to catalepsy assessment. Thirty, 60, 90, and 120 minutes following drug administration, the animal's forelimbs were draped over a thin horizontal rod 1¼″ high. The amount of time in seconds for which the animal maintained this awkward posture was recorded (60 seconds was maximum). Maintenance of this position was considered catalepsy. Animals that appeared to be sedated or had loss of muscle tone were checked for a righting reflex. Those mice that could not right themselves when placed on their backs were eliminated from the study. Mean time spent in the catalepsy position for the dose at each time was expressed as a percentage of maximum possible catalepsy. During the study's four time periods, the number of seconds with maintenance of the catalepsy position was recorded for each animal. Data was subjected to repeated-measures ANOVA followed by post-hoc analysis of least squares means. Males and females were analyzed separately.

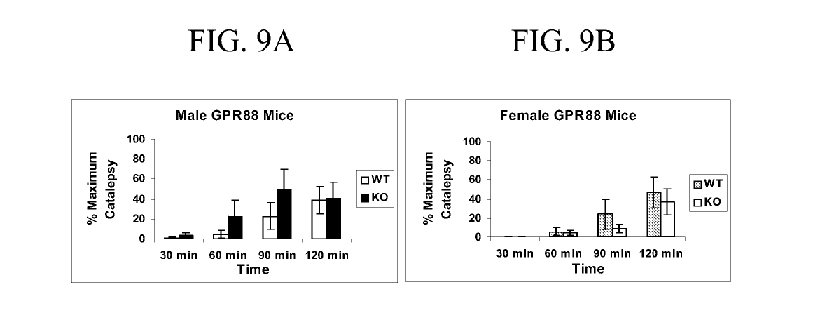

The results indicate that the male mice (FIG. 9A) did not exhibit a significant genotype effect (F(1, 10)=0.68, p>0.05), but there was a significant time effect (F(3, 30)=8.97, p<0.05) for both the GPR88 KO and WT male mice with the percentage of maximum catalepsy increasing to the 120 minute test point. There was no significant genotype/time interaction (F(3, 30)=1.03, p>0.05).

The profile was the same for the female mice (FIG. 9B). There was no significant genotype effect (F(1, 10)=0.58, p>0.05), but there was a significant time effect (F(3, 30)=9.48, p<0.05) with percentage of maximum catalepsy increasing to the 120 minute test point. There was no significant genotype/time interaction (F(3, 30)=0.34, p>0.05) and least squared means post-hoc analysis verified the lack of a difference between GPR88 KO and WT at any individual time point in both males and females (p>0.05).

No genotype or genotype/time interactions were observed between either female or male GPR88 KO and WT mice. For example, the 1 mg/kg dose of haloperidol provided equivalent levels of catalepsy in both the GPR88 KO and WT mice. Overall, the catalepsy assay results indicate that the GPR88 receptor does not affect the nigrostriatal system in comparison to the removal of the GPR88 receptor in the mesolimbic system.

### Example 9

### Measurement of Spontaneous Activity and Amphetamine-Stimulated Locomotor Hyperactivity

The amphetamine-stimulated locomotor hyperactivity assay is another type of assay to model the neuropathological states in schizophrenia, similar to the hyperdopaminergia in the ventral striatum. To measure spontaneous activity and amphetamine-stimulated locomotor hyperactivity, the mice were individually placed in Opto-Veramax-3 monitor cages (Columbus Instruments, Columbus, Ohio) and allowed to habituate for 30 minutes with room light and white noise on. After habituation, each mouse was injected with amphetamine (AMPH, 3 mg/kg) in the intraperitoneum. During the first 30 minutes, the spontaneous activity was recorded every 10 minutes. After amphetamine administration, amphetamine-stimulated locomotor hyperactivity was measured every 10 minutes over a 60 minutes time span by the activity monitor. Accuscan Versamax and Versadat software (Columbus Instruments, Columbus, Ohio) were used to convert the infrared beam breaks into horizontal activity counts. Activity counts every 10 minutes bins was analyzed by repeated-measures ANOVA followed by post-hoc analysis of least squares means. Males and females were analyzed separately.

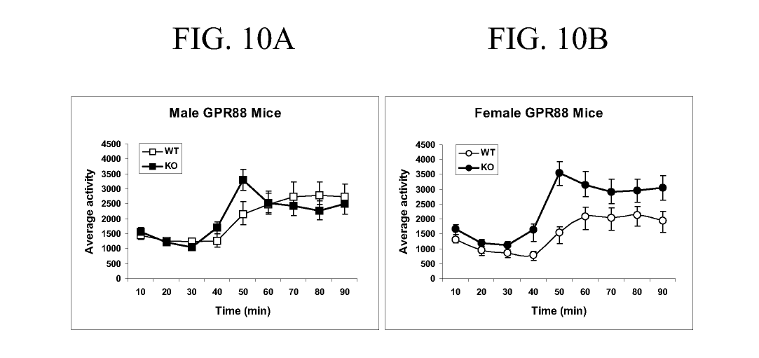

Results show that spontaneous activity in the male mice (FIG. 10A) was not significantly different between genotypes (F(1, 18)=0.08, p>0.05). However overall, there was a significant decrease in activity over the habituation period (0-30 min) (F(2, 36)=30.60, p<0.05), as well as a significant genotype/time interaction (F(2, 36)=4.92, p<0.05) with the WT mice not habituating to the same level by the end of the 30 minute habituation. Upon administration of amphetamine, there was a significant increase in activity of GPR88 KO male mice (F(5, 90)=12.28, p<0.05). After the observation of amphetamine-stimulated activity (40-90 min) was complete, however, there was not a significant genotype effect (F(1, 18)=0.04, p>0.05) between the GPR88 KO and WT male mice. There was also a significant genotype/time interaction (F(5, 90)=5.35, p<0.05) with the GPR88 KO male mice showing a greater increase in activity up to the 50 minute time point.

For the female mice (FIG. 10B), there was not a significant genotype effect during the habituation period (0-30 min) (F(1, 18)=2.54, p>0.05). However, there was a significant decrease in activity over the habituation period (F(2, 36)=57.82, p<0.05). There was no significant genotype/time interaction (F(2, 36)=0.94, p>0.05) for activity during habituation.

After amphetamine administration (40-90 min), the female mice did have a significant genotype effect (F(1, 18)=5.86, p<0.05) with the GPR88 KO female mice showing more hyperactivity overall as well as a significant increase in activity following amphetamine administration (F(5, 90)=18.84, p<0.05). There was also a significant genotype/time interaction (F(5, 90)=3.12, p<0.05) resulting from the GPR88 KO female mice demonstrating a larger increase in activity between the 40-50 minute period than was shown by the WT female mice.

### Example 10

### Measurement of Spontaneous Activity and Phencyclidine-Stimulated Locomotor Hyperactivity

The phencyclidine (PCP)-stimulated locomotor hyperactivity assay is used to model neuropathological states in schizophrenia. To measure PCP-stimulated locomotor hyperactivity, WT and GPR88 KO mice were individually placed in monitoring cages as described in Example 9, above, and allowed to habituate for 30 minutes with room light and white noise on (spontaneous activity). After habituation, each mouse received an intraperitoneal injection of PCP (0.1-0.3 mg/kg). During the first 30 minutes, the spontaneous activity was recorded every 10 minutes. After PCP administration, PCP-stimulated locomotor hyperactivity was measured every 10 minutes for a total of 60 minutes using a DIGISCAN™ animal activity monitor (Omnitech International, LTD, Midland, Mich.). ACCUSCAN™ Versamax and Versadat software (Columbus Instruments, Columbus, Ohio) were used to convert the infrared beam breaks into horizontal activity counts. Activity counts were analyzed in ten minute bins by repeated-measures ANOVA followed by post-hoc analysis of least squares means. Males and females were analyzed separately.

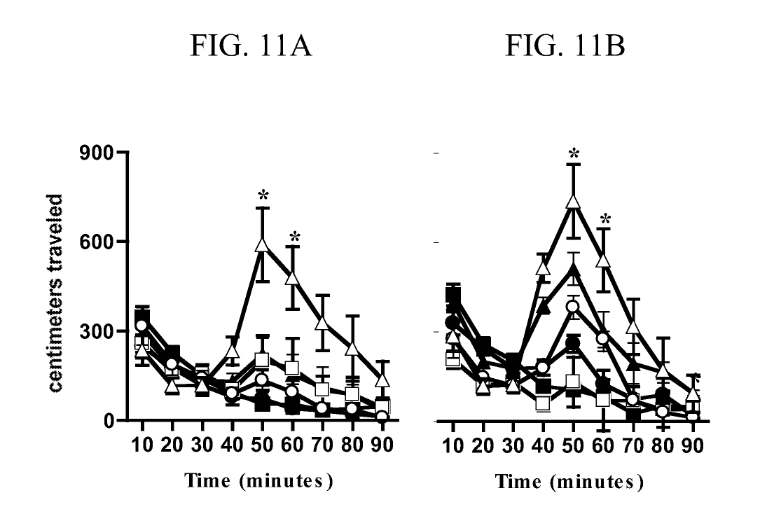

Results show that spontaneous activity in the male mice was not significantly different between WT and GPR88 KO genotypes (F(1, 61)=1.0, p>0.05). Upon administration of the highest PCP dose (3 mg/kg), increased locomotor activity was observed in both genotypes. Neither of the genotypes responded to lower doses of PCP. The GPR88 KO male mice demonstrated increased sensitivity for locomotor activation in comparison to the WT mice, as measured by the total distance traveled (FIG. 11A). A significant genotype/time interaction (F(8, 160)=3.92, p<0.0003) was also observed, with the GPR88 KO male mice showing a greater increase in activity 20-40 minutes after PCP administration.

For the female mice (FIG. 11B), there was a significant genotype effect during the habituation period (0-30 min), with GPR88 KO mice demonstrating hypoactivity (F(1, 59)=15.7, p<0.05). Upon administration of 1.7 mg/kg or 3.0 mg/kg of PCP, increased locomotor activity was observed in both genotypes. Neither of the genotypes responded to a lower dose of PCP (1.0 mg/kg). The GPR88 KO mice demonstrated increased sensitivity for locomotor activation, as measured by the total distance traveled. Significant genotype/time interactions at 1.7 mg/kg (F(8, 128)=2.26, p<0.027) and 3.0 mg/kg (F(8, 152)=2.65, p<0.01) were also observed, with the GPR88 KO female mice showing a greater increase in activity 20-30 minutes after PCP administration.

Similar to the results observed for the apomorphine-induced climbing and stereotypy model and the amphetamine-stimulated locomotor activity assay as described in Examples 6 and 9 above, respectively, the GPR88 KO mice were more sensitive to PCP-stimulated locomotor activity. Collectively, these assays indicate a combined behavioral profile of the GPR88 KO mice possessing a deficit in prepulse inhibition, increased sensitivity to apomorphine-induced climbing and stereotypy, and increased sensitivity to amphetamine stimulated and PCP-stimulated locomotor hyperactivity. This behavioral profile indicates that the GPR88 receptor is an element in the regulation of the dopamine system, and that deletion of the GPR88 receptor results in a mouse that exhibits a schizophrenia-like phenotype. The ability of antipsychotics such as haloperidol to normalize the prepulse inhibition deficit and block the apomorphine-induced climbing and stereotypy, further adds to the evidence that the GPR88 KO mouse is an accurate animal model for schizophrenia.

### Example 11

### Brain Structure of GPR88 KO Mice

In order to assess whether morphological abnormalities are associated with the observed schizophrenia-like behavioral phenotypes, the brain structure of the GPR88 KO mice was examined. Both WT and GPR88 KO mice were euthanized and their brains removed. Brains were collected (3 male GPR88 KO and 3 male WT) and rapidly frozen by burying in crushed ice. Tissues were sectioned at ten microns on a cryostat, mounted in series on Super Frost Plus slides (Fisher Scientific, Waltham, Mass.) and allowed to dry at room temperature for approximately 10 minutes. The slides were stored at −80° C. until use. The sections were stained with acetylcholinesterase or Niss1 according to standard protocols. Standard procedures for immunohistochemical staining were also followed for dopamine- and cyclic AMP-regulated phosphoprotein (DARPP-32), a marker of striatal medium spiny neurons.

The brains of the GPR88 KO mice showed no obvious cytoarchitectural abnormalities as assayed by examination of Niss1 stained sections. The cellularity and laminar pattern of the cortex seemed unperturbed by the loss of GPR88 and no variation in the primary somatosensory cortex barrel field was observed. Acetylcholinesterase staining through the striatum also did not reveal gross abnormalities to the caudatoputamen, nucleus accumbens, and the olfactory tubercle in GPR88 KO mice in comparison to WT. Immunohistochemical staining of DARPP-32 also did not reveal any variation of striatal medium spiny neurons between GPR88 KO and WT mice was observed.

### Example 12

### Density and Affinity of Striatal D₂ Receptors in GPR88 Mice

The GPR88 KO mice were also examined to assess whether the observed schizophrenia-like behavioral phenotypes were correlated with an increase in dopamine D₂ receptor density and/or affinity. Hietala et al. (Arch. Gen. Psychiatry, 1994, 57:116-23) have reported that the number and affinity of striatal dopamine D₂ receptors is not statistically different between neuroleptic-naïve schizophrenics and controls. However, Hietala et al. also observed a subgroup of patients with aberrant striatal D₂ dopamine receptor densities and affinity in vivo.

Striata from 10 mice (male and female, WT & GPR88 KO) were pooled and homogenized in a motor-driven POLYTRON™ homogenizer (Kinematica AG, Newark, N.J.) at setting six for 10 seconds in 3 mL per 100 mg of wet tissue in 50 mM Tris-HCl buffer, pH 7.4, containing 120 mM NaCl, 5 mM KCl, 1.5 mM CaCl₂, 4 mM MgCl₂, and 1 mM EDTA. The tissues were incubated with ten increasing concentrations of [³H]spiperone (0.005 nM to 5 nM), (GE Healthcare, Piscataway, N.J.). The binding of each concentration of radioligand was determined in duplicate. Incubations were carried out at room temperature for 120 minutes. The final concentration of the membrane protein in each binding assay was approximately 70 μg/ml. Butaclamol (+), (10 μM), (Sigma-Aldrich, St. Louis, Mo.) was used to define the nonspecific binding. The incubations were terminated by rapid filtration using a Wallac Tomtec harvester (Waltham, Mass.) and buffer-presoaked glass fiber filter mats (GF/B, Whatman Inc. Florham Park, N.J.). The filters were then rinsed with five 1 mL washes of cold 50 mM Tris buffer, pH 7.4, dried, and coated with MELTILEX™ B/HS scintillant sheets (Perkin Elmer Life and Analytical Sciences). The radioactivity of the filters was then counted in a Wallac 1205 scintillation counter (Perkin Elmer Life and Analytical Sciences).

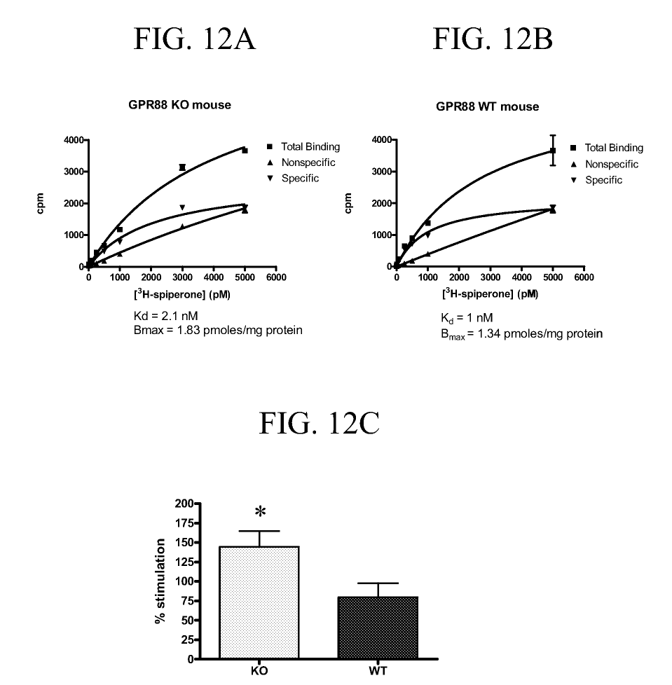

FIGS. 11A and 11B depict graphs of total binding, specific binding, and non-specific binding of [³H]spiperone for GPR88 KO (FIG. 12A) and WT (FIG. 12B) mice. Specific binding data were obtained for each data point by subtracting the mean of the nonspecific [³H]spiperone binding from the total binding mean. The estimates of Bₘₐₓ (the number of available binding receptors) and K_{d} (the dissociation constant) were calculated by fitting the specific binding data with nonlinear regression using GraphPad PRISM™ data analysis software (GraphPad Software, San Diego, Calif.). A Student's t-test was applied to the difference of the means to compare the groups.

Results show that the number of striatal dopamine D₂ receptors in the GPR88 KO mice was equivalent to that of the WT mice. (Bₘₐₓ=1.83 pmoles/mg for the GPR88 KO mice versus Bₘₐₓ=1.34 pmoles/mg protein for the WT mice). There was also no variation between groups for [³H]spiperone affinity (Kd=2.1 nM for the GPR88 KO mice versus Kd=1 nM for the WT mice).

### Example 13

### Percent Stimulation of [³⁵S]GTPγS Binding Due to Dopamine Agonism

GPR88 KO mice were further examined to determine if the dopamine D₂ receptors are more sensitive to dopamine activation than those of the WT mice. In order to determine dopamine sensitivity, dopamine stimulated [³⁵S]GTPγS binding was assessed in the striata of GPR88 KO and WT mice using the protocol essentially as described in Rinken et al. (Biochemical Pharmacology, 1999, 57: 155-162). Briefly, striata were collected from the mice (9 GPR88 KO or WT) and frozen at 80° C. until use. The tissue was homogenized in 1:100 volume (wet weight/volume) of ice-cold K-HEPES buffer (20 mM, pH=7.6), containing 7 mM of MgCl₂, 100 mM of NaCl, 1 mM of EDTA, and 1 mM of DTT (buffer A), and centrifuged at 45,000 g for 10 minutes at 4° C. The membrane pellet was resuspended in 100 volumes (w/v) of fresh buffer A and re-centrifuged under the same conditions.

The striatal membrane preparations (20 μl) were preincubated with guanosine 5′-diphosphate sodium salt (GDP) and dopamine (1 mM) for 30 minutes at 30° C. The incubation was started by addition of [³⁵S]GTPγS (final concentration 0.5 nM) and proceeded for 90 minutes at 30° C. The assay was terminated by filtration through a filter plate (GF/B, Perkin Elmer Life and Analytical Sciences) with five washings of cold buffer A (350 μl). The filter plate was air-dried and the radioactivity content of the filters was counted in 25 μl scintillation cocktail MICROSCINT™ 20 (Perkin Elmer Life and Analytical Sciences) by a Perkin Elmer TopCount NXT scintillation counter.

[³⁵S]GTPγS binding data were collected in triplicate for each individual sample in the presence and absence of dopamine. The data were transformed by log, and the mean of the triplicates for each sample in the presence and absence of dopamine was calculated. The percent stimulation of [³⁵S]GTPγS binding due to dopamine agonism was determined by calculating the difference of the mean binding between the presence and absence of dopamine. The percent stimulation [³⁵S]GTPγS binding for each genotype was then plotted as the mean of the individual values+/−the standard deviation using GraphPad PRISM™ (GraphPad) software. A Student's t-test was applied to the difference of the means to compare the groups. The results show that dopamine D₂ receptors in GPR88 KO mice were more sensitive to dopamine activation than the D₂ receptors of WT mice. The stimulation of D₂ receptors in GPR88 KO mice was twice the amount of stimulation observed in WT mice (FIG. 12C).

### Example 14

### Ex Vivo Neurochemical Characterization of Striatal Tissue in GPR88 KO Mice

The schizophrenia-like phenotype identified by behavioral assays can be further strengthened by an assessment of the neurochemical profile of dopamine in the striatum to help identify the specific role of GPR88 receptor in the striatal dopamine system. Accordingly, an ex vivo neurochemical characterization of dopamine levels in the striatal tissue of the GPR88 mice (male and female, WT and KO) was performed.

Both WT and GPR88 KO male and female mice were euthanized by exposure to CO₂ and their brains removed. Striatal tissue was dissected on ice and frozen for subsequent analysis. Frozen striatal tissue samples were weighed and perchloric acid (0.4M) added at 10 μl/mg tissue. Samples were homogenized using a sonicating probe. Samples were then centrifuged at 15,000 rpm for 20 minutes and the supernatant removed. The supernatant was then centrifuged in a filtered tube at 3,000 rpm for 5 minutes. This supernatant was then diluted 1:100 to measure the concentration of dopamine and 1:10 to measure the concentration of the dopamine metabolites DOPAC (3,4-dihydroxyphenylacetic acid) and homovanillic acid (HVA). Supernatant was analyzed for neurochemical content using high performance liquid chromatography electrochemical detection system (HPLC-ECD).

Chromatographic separations were performed using reverse phase HPLC (C18 ODS3 column, 150×3.0 mm, Metachem, Torrance, Calif., USA). The mobile phase comprised 0.15 M NaH₂PO₄, 0.25 mM EDTA, 1.75 mM 1-octane sulphonic acid, 2% isopropanol and 4% methanol, pH=4.6 and delivered at 0.5 ml/minutes at 30° C. Dopamine and the dopamine metabolites were detected using an electrochemical amperometric detector (Decade, Antec-Leyden, NL) with a working electrode set at +650 mV vs Ag/AgCl reference electrode.

Results show that concentrations of dopamine were the same in all groups and there was no effect of gender or genotype on the levels of dopamine (Table 1). As expected, levels of the DOPAC and HVA were lower than dopamine, but there was no difference between the WT and GPR88 KO mice, male or female (Table 1).

TABLE 1
Concentration of dopamine, DOPAC and HVA in striatal tissue
in male and female GPR88 wild type and knockout mice. Data
are mean concentration ± sem (n = 6; μM).

| | Male | | Female | |
|---|---|---|---|---|
| Neurochemical | Wild type | GPR88 KO | Wild type | GPR88 KO |
| Dopamine | 89.5 ± 8.0 | 75.2 ± 10.8 | 82.1 ± 8.8 | 82.6 ± 7.5 |
| DOPAC | 2.1 ± 0.3 | 2.0 ± 0.4 | 2.1 ± 0.4 | 2.3 ± 0.3 |
| HVA | 2.2 ± 0.3 | 1.8 ± 0.4 | 2.3 ± 0.2 | 2.1 ± 0.3 |

### Example 15

### In Vivo Striatal Microdialysis of GPR88 Mice

High levels of striatal dopamine have been correlated with poor performance in prepulse inhibition of starte (Goto et al., Psychiatry and Clinical Neurosciences 58:441-445 (2004)). In addition, an indirect relationship can be made in schizophrenic patients who suffer from disrupted prepulse inhibition and an increased level of striatal dopamine (Abi-Dargham and Laruelle, European Psychiatry: Journal of the Association of European Psychiatrists 20:15-27 (2005); Braff et al., Psychopharmacology 156:234-258 (2001)). To test whether the schizophrenia-like behavioral phenotypes was due to an increased level of striatal dopamine in vivo, levels of striatal dopamine levels were tested in the GPR88 KO mice in vivo.

GPR88 KO and WT mice (male and female) were anesthetized with ketamine and DOMITOR® and were implanted with microdialysis guide cannulae (CMA7) above the striatum (AP+0.5; ML−2.0; DV−1.6 mm). The guide and a tether pin were secured using dental acrylic and 2 small skull screws. Anesthesia was reversed using ANTISEDAN® and mice were administered buprenorphine for analgesia. After surgery mice were singly housed. At least 3 days later, mice were placed in microdialysis cages the afternoon prior to microdialysis experiments. On the day of the experiment, mice were briefly anesthetized with halothane to allow removal of the obturator from the guide and insertion of the probe (CMA 7, cpr/02) with the tether attachment. Probes were perfused with artificial CSF at 1 μl/minutes. After a 3-hour habituation, 4 basal samples were collected every 30 minutes. Mice were then dosed intraperitoneally with vehicle (saline, 10 ml/kg) or amphetamine (3 mg/kg). Dialysates were collected for 3 hours after administration of drug. After completion of the collection period, probes were removed from guide cannulae (under manual restraint) and obturator was replaced. Mice were returned to their home cages and re-used in a cross-over design 1 week later. Dialysates were analyzed for dopamine content using HPLC-ECD. After the second experiment, mice were euthanized and the brains removed for histological verification of probe placement.

Chromatographic separations were performed using a Capcell PAK™, Strong Cation Exchange columns (5 μm UG80, 1.5×150 mm; Shiseido, Phenomenex, USA). The mobile phase comprised (mM) 13 Na₂HPO₄, 87 NaH₂PO₄, 0.1 EDTA and 20% methanol, was adjusted to pH 6 and delivered at 0.2 ml/minutes at 40° C. Dopamine was detected using an electrochemical amperometric detector (Decade II, Antec-Leyden, NL) with a working electrode set at +500 mV vs Ag/AgCl reference.

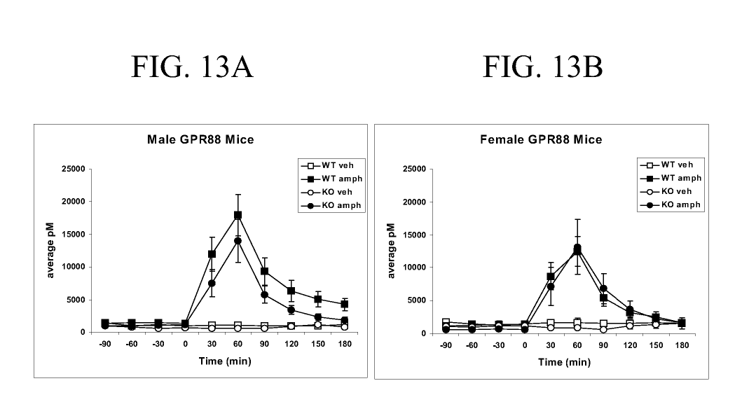

Table 2 shows that baseline concentrations of extracellular dopamine were significantly lower in GPR88 KO than WT mice (F(1, 54)=4.37, p<0.05). Administration of amphetamine caused a significant and equal increase in dopamine in both genotypes and in both males (FIG. 13A) and females (FIG. 13B), (F(7, 48)=8.88, p<0.05).

TABLE 2
Baseline dialysate concentrations of striatal dopamine
in male and female WT and GPR88 KO mice. Data are mean
concentration ± sem (n = 13-14; pM).

| Male | | Female | |
|---|---|---|---|
| Wild type | KO | Wild type | KO |
| 1194.8± 210.6 | 886.2 ± 125.4 | 1260.6 ± 308.2 | 740.3 ± 142.3 |

Interestingly, the GPR88 KO and WT mice had similar levels of dopamine and dopamine metabolites in the striatum. This result indicates that mechanisms other than total dopamine levels underlie the schizophrenia-like phenotype of the GPR88 KO mice.

One alternative mechanism is that the amount of dopamine released into the extra-cellular space upon stimulation of the dopaminergic neurons is greater in GPR88 KO mice than in the wild type mice. This alteration in the dopamine system would fit with the increased response to amphetamine in the amphetamine-stimulated locomotor hyperactivity assay and would parallel the increased release of dopamine in response to amphetamine has been seen in schizophrenic patients. However, using in vivo striatal microdialysis, the comparison of GPR88 KO mice to wild type mice showed that the GPR88 KO mice had lower baseline levels of dopamine in extracellular spaces, but there was no difference between the GPR88 KO and wild type mice in the amount of dopamine released in response to a challenge with 3 mg/kg of amphetamine. In response to amphetamine, the lack of increased dopamine release in the striatum indicates that increased presynaptic function of the DA system is not the underlying mechanism for the disrupted PPI or the increased sensitivity to amphetamine-stimulated locomotor activity in the GPR88 KO mice. In conjunction with the lack of potentiated dopamine release in the striatum, the lower basal level of dopamine in extracellular spaces indicates an increased sensitivity of the D2 receptors in the postsynaptic neurons that may underlie the schizophrenia-like behavioral phenotype in the GPR88 KO mice.

### Example 16

### Evaluation of Spatial and Cued Learning Using Conditioned Fear in GPR88 Mice

The conditioned fear assay is a simple measure of cognitive function using spatial cues and a single auditory cue. Schizophrenic patients are known to have severe deficits in numerous cognition domains especially those involved in more complex tasks. An animal model with a schizophrenia-like cognition phenotype should be impaired in complex learning tasks (i.e., spatial or context conditioned fear) while simple rule learning (i.e., cued fear conditioning) should be intact. To measure context and cued conditioned fear, the mice were placed in conditioning chambers (MED Associates of St. Albans, Vt.) on training day and after 2 minutes they were exposed to a 30-second tone that was paired with a 2-second (0.7 mA) footshock. Another tone shock pairing was given 2 minutes after the first pair and the mice were removed from the chamber 30 seconds after a second shock. Twenty-four hours later the mice were returned to the training chamber and scored for freezing (e.g., a complete lack of movement except for respiration) every 10 seconds over a 5-minute test session to measure the amount of contextual conditioned fear. Approximately 2 hours later, the mice were returned to chamber which had been altered by the addition of a Plexiglas insert which provided a smooth floor surface and reduced the size of the chamber to one-half the original size with a new triangular configuration (e.g., the altered context). Cued conditioned fear was assessed by scoring the mice for freezing every 10 seconds for a total duration of six minutes with re-exposure to the tone occurring during the final 3 minutes of the test session. The data was converted to the percentage of 10-second freezing periods with respect to the entire test session. The percentage freezing for context, altered context and cue was analyzed with ANOVA with the genotype as the factor and the ANOVA was followed by post-hoc analysis of least squares means. Males and females were analyzed separately. A high percentage of the test time spent freezing is indicative of good learning and memory.

Table 3 shows the results for the male mice. The GPR88 KO mice showed a nearly significant decrease in freezing to the context (F(1, 23)=3.956, p=0.059) compared to the WT mice. When tested in the altered context, the GPR88 KO mice showed freezing equal to the WT mice in the altered context (F(1, 23)=1.034, p>0.05) and to the tone cue (F(1, 23)=3.475, p>0.05).

Unlike with the male mice, the GPR88 KO female mice showed equal freezing to the context (F(1, 24)=0.771, p>0.05) compared to the WT mice (Table 3). When tested in the altered context, the GPR88 KO mice again showed equal freezing relative to the WT mice in the altered context (F(1, 24)=2.701, p>0.05) as well as equal freezing to the tone cue (F(1, 24)=0.027, p>0.05).

TABLE 3
Conditioned Fear in male and female GPR88 wild type and knockout
mice. Data are mean percentage freezing ± sem (n = 12-13).

| | Male | | Female | |
|---|---|---|---|---|
| Test | Wild type | GPR88 KO | Wild type | GPR88 KO |
| Context | 65.6 ± 6.6 | 45.6 ± 7.6 | 59.7 ± 8.8 | 49.5 ± 7.7 |
| Altered Context | 49.1 ± 8.0 | 37.5 ± 8.1 | 47.2 ± 7.8 | 31.2 ± 5.9 |
| Cue | 95.4 ± 1.8 | 89.4 ± 2.7 | 90.7 ± 3.5 | 91.5 ± 2.6 |

In male GPR88 KO mice, there was a learning deficit in the complex/spatial component of conditioned fear while the simple/cue learning was unaffected. This profile is similar to the cognitive impairment that is characteristic in schizophrenia. The lack of cognitive impairment in the female GPR88 KO mice may be attributable to a delayed onset of the schizophrenia-like cognition phenotype as has been reported human female patients (Harris et al., Schizophr. Bull. 14:39-55 (1988).

While the present invention has been described in terms of the certain embodiments, it is understood that variations and modifications will occur to those skilled in the art. Therefore, it is intended that the appended claims cover all such equivalent variations that come within the scope of the invention as claimed.

What is claimed is:

1. A method of identifying an agent for the treatment of a schizophrenia-like phenotype, said method comprising:
(a) administering a test agent to a transgenic animal lacking GPR88 function and exhibiting a schizophrenia-like phenotype;
(b) measuring a response of the transgenic animal to the test agent; and
(c) determining whether the test agent reduces or enhances the schizophrenia-like phenotype.

2. The method of claim 1, wherein the agent identified is an agonist.

3. The method of claim 1, wherein the agent identified is an antagonist.

4. A method of treating a human subject suffering from schizophrenia, wherein the human subject is treated with the agonist of claim 2.

5. A method of identifying an agent for the treatment of a neurological disorder affecting dopamine D2 receptors, said method comprising:
(a) administering a test agent to a transgenic animal lacking GPR88 function and having enhanced D2 receptor sensitivity to dopamine stimulation;
(b) measuring a response of the transgenic animal to the test agent; and
(c) determining whether the test agent reduces or enhances dopamine D2 receptor sensitivity.

6. The method of claim 5, wherein the neurological disorder is selected from a group consisting of schizophrenia, Parkinson disease, Huntington disease, attention deficit hyperactivity disorder (ADHD), hallucination induced by drug abuse or epilepsy, Tourette's syndrome, restless leg syndrome, obsessive-compulsive disorder (OCD), mania, depression, addiction, or hyperphagia.

7. The method of claim 5, wherein the agent identified is an agonist.

8. The method of claim 5, wherein the agent identified is an antagonist.

9. A method of identifying an agent for the treatment of a schizophrenia-like phenotype, said method comprising:
(a) administering a test agent to a transgenic animal lacking GPR88 function and exhibiting a schizophrenia-like phenotype comprising (i) a deficit in prepulse inhibition, (ii) an increased sensitivity to apomorphine-induced behaviors, (iii) a decreased level of extracellular dopamine in the striatum, or a combination of any of (i)-(iii);
(b) measuring a response of the transgenic animal to the test agent; and
(c) determining whether the test agent reduces or enhances the schizophrenia-like phenotype.

10. The method of claim 9, wherein the agent identified is an agonist.

11. The method of claim 9, wherein the agent identified is an antagonist.

12. A method of treating a human subject suffering from a schizophrenia-like phenotype, wherein the human subject is treated with the agonist of claim 10.
